(19) United States
(12) Patent Application Publication
(10) Pub. No.: US 2026/0240740 A1
(43) Pub. Date: Aug. 20, 2026

(54) MULTI-PHOTOPROTECTIVE COMPOUNDS AND USES THEREOF

(71) Applicant: SKINOSIVE, PARIS (FR)

(72) Inventors: ISABELLE RAULT, GÂVRES (FR); DENIS CARNIATO, MARCOUSSIS (FR); CÉDRIC YSACCO, SAINT-PAUL-TROIS-CHÂTEAUX (FR); JULIETTE BERTRAND, MARSEILLE (FR)

(21) Appl. No.: 18/876,690

(22) PCT Filed: Jun. 21, 2023
(86) PCT No.: PCT/EP2023/066739

§ 371 Date: Dec. 19, 2024

(30) Foreign Application Priority Data
Jun. 22, 2022 (EP) 22305904.9

(51) Int. Cl. A61K 8/49; A61Q 17/04; C07C 255/41; C07D 251/22; C07D 403/12

(52) U.S. Cl. CPC A61K 8/4966; C07C 255/41; C07D 251/22; C07D 403/12; A61Q 17/04

(57) ABSTRACT

The present invention relates to a compound represented by the following formula (I): [(A)ₙ-B₁]ₘ(X)ₓ[B₂—(C)_{n′}]_{m′} (I), wherein X is a radical derived from an aryl, a heteroaryl, a polyol, a saccharide, an amino-acid, a peptide, or a polyamine; each A represents independently a photoprotective moiety, each B₁ and B₂ represents independently a linker, C represents a functional group, x is 0 or 1, n is an integer from 1 to 12, n′ is an integer from 1 to 2000, m is an integer from 1 to 12, m′ is an integer from 0 to 12, and the number of photoprotective moieties A (m×n) is superior or equal to 2. It also relates to a composition comprising the same and, more particularly, to a cosmetic or a sunscreen composition. The invention further relates to a material comprising a support and such a compound adhered to said support.

### TECHNICAL FIELD

The present invention relates to the field of UV filters. More particularly, the invention relates to compounds comprising a plurality of photoprotective moieties. It also relates to a composition comprising the same and uses thereof in cosmetic applications. The invention further relates to a material comprising a support and a compound of the invention.

### TECHNICAL BACKGROUND

Almost 5% of the electromagnetic energy of the sun is emitted in the form of UV light. This UV light can be divided into three groups: UV-A (400-315 nm), UV-B (315-280 nm) and UV-C (280-100 nm). UV light, and more particularly UV-B light, can have damaging short-term or long-term effects on the body. Serious skin damages may occur due to an exposure to UV light, such as an accelerated skin ageing or a skin cancer. As a result, the development of new sunscreens providing effective protection against UV radiation has become a prime concern. More than thirty mineral and organic UV filters, including homosalate, ecamsule, or octocrylene, are nowadays allowed in cosmetic products and available on the market.

However, such sunscreens used alone have a narrow protection spectrum and need to be applied with large amounts on the skin several times in a day, to ensure an efficient UV skin protection.

Sunscreen compositions comprising a mixture of UV-A and UV-B filters have been proposed to enlarge the skin protection. However, higher concentrations of UV filters are usually required in such compositions, which is not desirable. Moreover, such mixtures do not allow a high homogeneity in terms of covering on the skin surface.

Thus, there remains a need to develop further photoprotective compounds having an improved photoprotection.

### SUMMARY OF THE INVENTION

In this respect, the inventors have developed new efficient photoprotector compounds. Such compounds comprise at least two photoprotective moieties, linked to each other through a linker, and/or each linked to a central core through a linker. In a particular embodiment, the compounds may comprise at least one functional group, which make them adhesive or bioadhesive, improving thereby their efficacy for UV protection, when applied to a support, in particular the skin.

Thus, the present invention relates to a compound represented by the following formula (I):

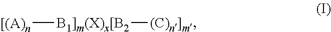

wherein:
X is a radical derived from an aryl, a heteroaryl, a polyol, a saccharide, an amino-acid, a peptide, or a polyamine;
each A represents independently a photoprotective moiety,
each B₁ and B₂ represents independently a linker,
C represents a functional group,
x is 0 or 1,
n is an integer from 1 to 12,
n′ is an integer from 1 to 2000,
m is an integer from 1 to 12,
m′ is an integer from 0 to 12, and
the number of photoprotective moieties A (m×n) is superior or equal to 2.

Preferably, each photoprotective moiety A is independently derived from bemotrizinol, diethylamino hydroxybenzoyl hexyl benzoate, bisdisulizole disodium, meradimate, terephtalylidene dicamphor sulfonic acid, oxybenzone, sulisobenzone, iscotrizinol, octinoxate, octisalate, octyltriazone, padimate O, homosalate, amiloxate, octocrylene, PEG-25 PABA, ensulizole, trolamine salicylate, cinoxate, benzophenone-9, dioxybenzone, avobenzone, enzacamene, bisoctrizole, diethylhexyl naphthalate, diethylhexyl syringylidene, tetramethylhydroxy piperidinol, sodium benzotriazolyl butylphenol sulfonate, benzotriazole dodecyl p-cresol sulfonate, butyloctyl salicylate, bis(cyano butylacetate) anthracenediylidene, dimethylcapramide, ethyl hexyl methoxycrylene, ethyl hexyl triazone, 2,4,6-tri([1,1′-biphenyl]-4-yl)-1,3,5-triazine, 2-(4,6-diphenyl-1,3,5-triazin-2-yl)-5-[(hexyl)oxy]-phenol, 2,4-Bis(2,4-dimethylphenyl)-6-(2,4-dihydroxyphenyl)-1,3,5-triazine, 4-(4,6-Bis((biphenyl-4-yl)-1,3,5-triazine-2-yl)-1,3-benzodiole, more preferably derived from diethylamino hydroxybenzoyl hexyl benzoate, butyloctyl salicylate, bemotrizinol, terephtalylidene dicamphor sulfonic acid, homosalate, octocrylene, ensulizole, octisalate, ethyl hexyl triazone, 2-(4,6-diphenyl-1,3,5-triazin-2-yl)-5-[(hexyl)oxy]-phenol, 2,4-Bis(2,4-dimethylphenyl)-6-(2,4-dihydroxyphenyl)-1,3,5-triazine, or 4-(4,6-Bis((biphenyl-4-yl)-1,3,5-triazine-2-yl)-1,3-benzodiole.

In a particular embodiment, the compound of formula (I) is such that:
x is 1, and
m′ is an integer from 1 to 12.

In a particular embodiment, each functional group C is independently chosen from an aldehyde, an acetal, thiocetal, a thiol, a maleimide, a 5-methylene pyrrolone, a 3-bromo-5-methylene pyrrolone, a 5-hydroxy-pyrrolone, a 3,3-dimethylacrylic acid, a Mickael acceptor, a vinylsulfone, a disulfanylpyridine, a sulfonylaziridine, a vinylheteroarene, a vinylpyridine, a vinylpyrimidine, a vinyltriazine, a vinyltetrazine, an epoxide, a haloacetyl, an isocyanate, an isothiocyanate, a N-hydroxysuccinimide ester, a N-hydroxysulfosuccinimide ester, a hydroxy, an amino, an ammonium, a guanidinium, an imidocarbonate, a carboxylic acid, a carboxylic ester, an anhydride, a sulfonic acid, folic acid, biotin, streptavidin, avidin, antibodies, and single chain antibodies or fragments thereof, and derivatives thereof, preferably from a maleimide, a 5-methylene pyrrolone and derivatives thereof.

In another particular embodiment, the compound of formula (I) is such that:
x is 1, and
m′ is 0,
provided that at least two photoprotective moieties A are different.

In a particular embodiment, said radical X is derived from a phenyl, a triazinyl, a glycerol or a pentaerythritol.

In another particular embodiment, the compound of formula (I) is such that:
x is 0, and
m′ is 0,
   provided that at least two photoprotective moieties A are different.

In a particular embodiment, the linkers B₁ and B₂ are each independently a saturated or unsaturated, linear or branched carbon chain, an aliphatic or aromatic carbocycle, an aliphatic or aromatic heterocycle, a linear polymer, a branched polymer, a hyperbranched polymer, a dendrimer, or a residue thereof, and said linker optionally comprises at any one or both of its terminals a heteroatom independently chosen from —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—.

The linkers B₁ and B₂ may each independently be interrupted by:
one or more heteroatomic groups chosen each independently from —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—, and/or
one or more heterocycloalkyls, such as a tetraoxaspiro[5.5]undecanyl.

In another particular embodiment, the linkers B₁ and B₂ are each independently of formula (II):

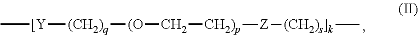

wherein:
Y₁ is selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—;
q is an integer from 0 to 35, preferably from 0 to 12, more preferably from 0 to 6;
with the proviso that, when Y is —O—, q is different from 0;
p is an integer from 0 to 250, preferably from 0 to 50, more preferably from 0 to 12; and
p+q is different from 0; or
Y₁—(CH₂)_{q} may form a heterocycle selected in the group consisting of a pyrrolidinyl and piperidinyl; and
p is 0;
Z₁ is selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—;
s is an integer from 0 to 6; and
k is an integer from 1 to 4, preferably from 1 to 2.

In another particular embodiment, the linkers B₁ and B₂ are each independently of formula (III):

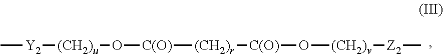

in which,
Y₂ and Z₂ are each independently selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—;
u and v are independently an integer from 1 to 50, preferably from 1 to 10, more preferably from 1 to 5, preferably u and v are independently 2, 3, or 4, and
r is an integer from 1 to 28, preferably r is 4.

In another particular embodiment, the linkers B₁ and B₂ are each independently of formula (IV):

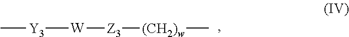

Y₃ and Z₃ are each independently selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—;
W is an aryl or a heteroaryl; and
w is an integer from 1 to 150, preferably from 1 to 50, more preferably from 1 to 10.

In a preferred embodiment, each linker B₂ is independently represented by one of the following formulae:

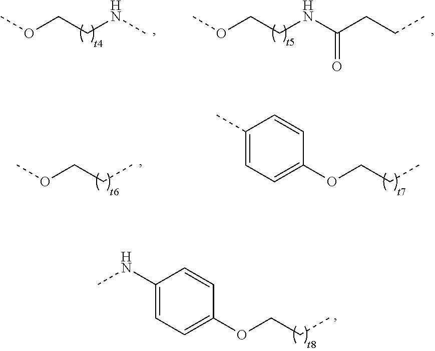

in which t4, t5, t6, t7, and t8 are each independently an integer from 0 to 30, preferably from 0 to 12.

In a more preferred embodiment, each moiety —B₂—(C)ₙ′ is independently represented by one of the following formulae:

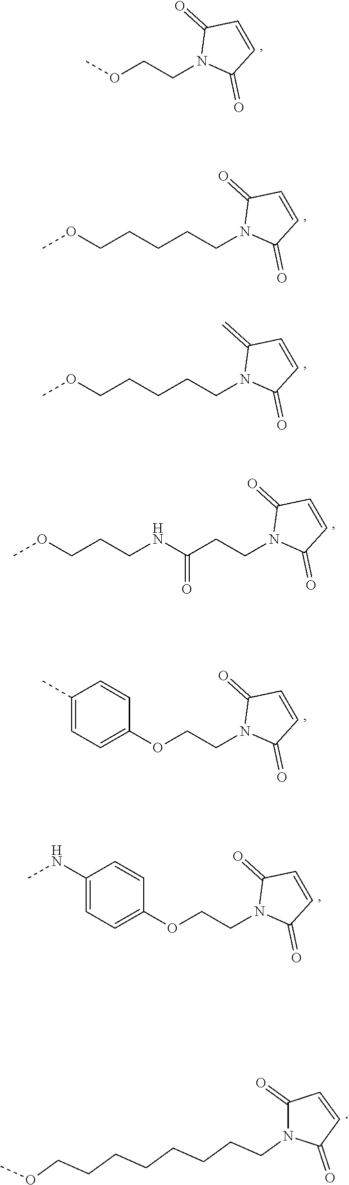

Preferably, said compound is selected from the group consisting of:
2-[(2-cyano-3,3-diphenyl-prop-2-enoyl)amino]ethyl 2-[4-(diethylamino)-2-hydroxy-benzoyl]benzoate;
8-[[2-[4-(diethylamino)-2-hydroxy-benzoyl]benzoyl]amino]octyl 2-cyano-3,3-diphenyl-prop-2-enoate;
8-[4-(4,6-diphenyl-1,3,5-triazin-2-yl)-3-hydroxy-phenoxy]octyl 2-hydroxybenzoate;
7-[4-[4,6-bis[4-(2-ethylhexoxy)-2-hydroxy-phenyl]-1,3,5-triazin-2-yl]phenoxy]heptyl 2-(4-amino-2-hydroxy-benzoyl)benzoate;
8-[4-(4,6-diphenyl-1,3,5-triazin-2-yl)-3-hydroxy-phenoxy]octyl 2-cyano-3,3-diphenyl-prop-2-enoate;
2-[2-[2-[4-[4,6-bis[4-(2-ethylhexoxy)-2-hydroxy-phenyl]-1,3,5-triazin-2-yl]phenoxy]ethoxy]ethoxy]ethyl 2-cyano-3,3-diphenyl-prop-2-enoate;
N-[7-[4-[4,6-bis[4-(2-ethylhexoxy)-2-hydroxy-phenyl]-1,3,5-triazin-2-yl]phenoxy]heptyl]-2-hydroxy-benzamide;
6-[[2-[4-(diethylamino)-2-hydroxy-benzoyl]benzoyl]amino]hexyl 2-hydroxybenzoate;
5-[5-(2-ethylhexoxy)-2-[4-[4-(2-ethylhexoxy)-2-hydroxy-phenyl]-6-(4-methoxyphenyl)-1,3,5-triazin-2-yl]phenoxy]pentyl 2-cyano-3,3-diphenyl-prop-2-enoate
N-[5-[4-[4,6-bis(2,4-dimethylphenyl)-1,3,5-triazin-2-yl]-3-hydroxy-phenoxy]pentyl]-2-[4-(diethylamino)-2-hydroxy-benzoyl]benzamide;
[(3Z)-3-[[4-[(Z)-[4-[7-[2-(2-hydroxy-4-methyl-benzoyl)benzoyl]oxyheptylsulfamoylmethyl]-7,7-dimethyl-3-oxo-norbornan-2-ylidene]methyl]phenyl]methylene]-7,7-dimethyl-2-oxo-norbornan-1-yl]methanesulfonic acid;
N-[5-[4-[4,6-bis(4-phenylphenyl)-1,3,5-triazin-2-yl]-3-hydroxy-phenoxy]pentyl]-2-[4-(diethylamino)-2-hydroxy-benzoyl]benzamide;
5-[(2-phenyl-3H-benzimidazol-5-yl)sulfonylamino]pentyl-2-[4-(diethylamino)-2-hydroxy-benzoyl]benzoate:
4-[9-[6-(2-cyano-3,3-diphenyl-prop-2-enoyl)oxyhexyl]-2,4,8,10-tetraoxaspiro[5.5]undecan-3-yl]butyl 2-[4-(diethylamino)-2-hydroxy-benzoyl]benzoate;
[(3Z)-3-[[4-[(Z)-[4-[3-[6-[2-(2-hydroxybenzoyl)oxyethoxy]-6-oxo-hexanoyl]oxypropylsulfamoylmethyl]-7,7-dimethyl-3-oxo-norbornan-2-ylidene]methyl]phenyl]methylene]-7,7-dimethyl-2-oxo-norbornan-1-yl]methanesulfonic acid;
O6-[3-(2-cyano-3,3-diphenyl-prop-2-enoyl)oxypropyl]O1-[3-[2-[4-(diethylamino)-2-hydroxy-benzoyl]benzoyl]oxypropyl]hexanedioate;
[(3Z)-3-[[4-[(Z)-[4-[3-[6-[2-[(5-benzoyl-4-hydroxy-2-methoxy-phenyl)sulfonylamino]ethoxy]-6-oxo-hexanoyl]oxypropylsulfamoylmethyl]-7,7-dimethyl-3-oxo-norbornan-2-ylidene]methyl]phenyl]methylene]-7,7-dimethyl-2-oxo-norbornan-1-yl]methanesulfonic acid;
2-[[4,6-bis[4-(2-cyano-3,3-diphenyl-prop-2-enoyl)oxybutylamino]-1,3,5-triazin-2-yl]amino]ethyl-2-(2-hydroxy-4-methyl-benzoyl)benzoate;
2-[[4-[2-(2,5-dioxopyrrol-1-yl)ethoxy]-6-[2-[(2-hydroxybenzoyl)amino]ethylamino]-1,3,5-triazin-2-yl]amino]ethyl 2-[4-(diethylamino)-2-hydroxy-benzoyl]benzoate;
N-[2-[[4-[5-(2,5-dioxopyrrol-1-yl)pentoxy]-6-[2-[2-[2-[2-[[rac-(3Z,4R)-7,7-dimethyl-2-oxo-3-[[4-[rac-(Z)-[rac-(1R)-4-ethyl-7,7-dimethyl-3-oxo-norbornan-2-ylidene]methyl]phenyl]methylene]norbornan-1-yl]methylsulfonylamino]ethoxy]ethoxy]ethoxy]ethylamino]-1,3,5-triazin-2-yl]amino]ethyl]-2-hydroxy-benzamide;
2-hydroxy-N-[2-[[4-[5-(2-methylene-5-oxo-pyrrol-1-yl)pentoxy]-6-[2-[2-[2-[2-[[rac-(3Z,4R)-7,7-dimethyl-2-oxo-3-[[4-[rac-(Z)-[rac-(1R)-4-ethyl-7,7-dimethyl-3-oxo-norbornan-2-ylidene]methyl]phenyl]methylene]norbornan-1-yl]methylsulfonylamino]ethoxy]ethoxy]ethoxy]ethylamino]-1,3,5-triazin-2-yl]amino]ethyl]benzamide;
2-[[4-[4-[2-(2,5-dioxopyrrol-1-yl)ethoxy]anilino]-6-[2-[(2-hydroxybenzoyl)amino]ethyl amino]-1,3,5-triazin-2-yl]amino]ethyl 2-[4-(diethylamino)-2-hydroxy-benzoyl]benzoate;
2-[[4-[4-[2-(2,5-dioxopyrrol-1-yl)ethoxy]phenyl]-6-[2-[(2-hydroxybenzoyl)amino]ethyl amino]-1,3,5-triazin-2-yl]amino]ethyl 2-[4-(diethylamino)-2-hydroxy-benzoyl]benzoate;
6-[[2-[4-(diethylamino)-2-hydroxy-benzoyl]benzoyl]amino]hexyl 2-cyano-3,3-diphenyl-prop-2-enoate;
2-cyano-N-(8-(5-((2-ethylhexyl)oxy)-2-(4-(4-((2-ethylhexyl)oxy)-2-hydroxyphenyl)-6-(4-methoxyphenyl)-1,3,5-triazin-2-yl)phenoxy)octyl)-3,3-diphenylacrylamide and
2-cyano-N-(8-(2-(4-(2-((8-(2,5-dioxo-2,5-dihydro-1H-pyrrol-1-yl)octyl)oxy)-4-((2-ethylhexyl)oxy)phenyl)-6-(4-methoxyphenyl)-1,3,5-triazin-2-yl)-5-((2-ethylhexyl)oxy)phenoxy)octyl)-3,3-diphenylacrylamide.

Still more preferably, said compound has one of the following formulae:

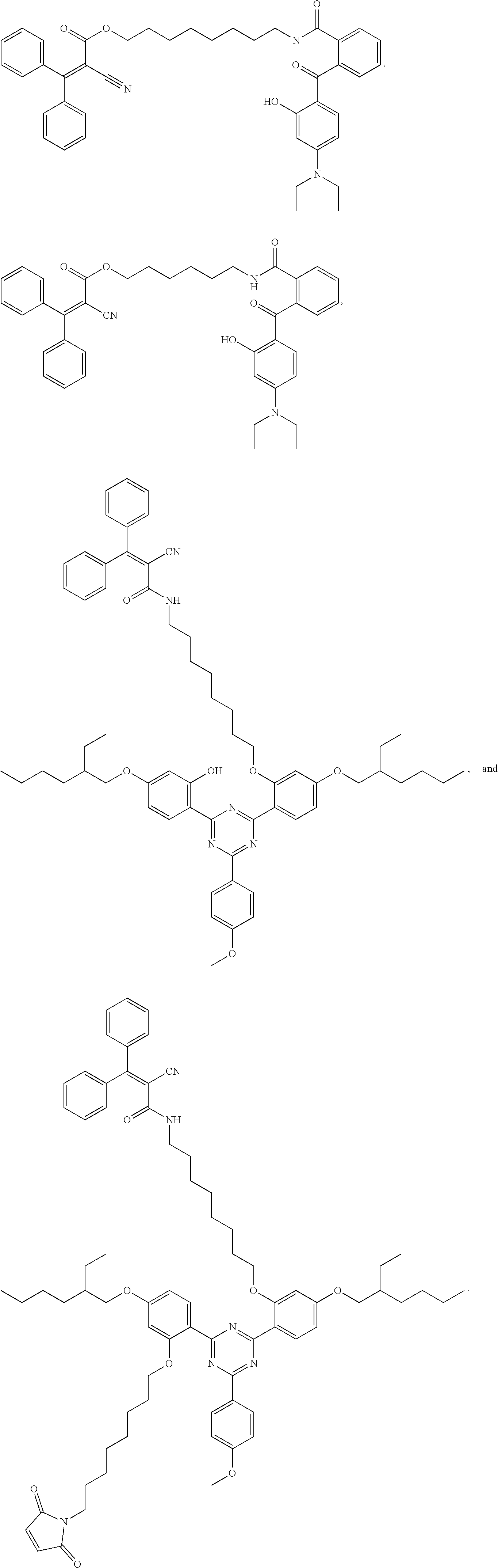

Another object of the invention is a composition comprising at least one compound of formula (I) as defined herein and at least one excipient.

In a particular embodiment, said composition is a sunscreen composition or a cosmetic composition.

In another particular embodiment, said composition is a pharmaceutical or a veterinary composition.

Said composition may in particular be is a topical composition, preferably said composition is in the form of a suspension, a cream, a spray, an aerosol, a butter, a stick, a gel, an ointment, a lotion, a solution, a solid, an emulsion, a micro-emulsion, an oil, a lyophilizate, a milk, a powder, a paste, a wax, a mousse, a patch, a film, a micelle, a liposome, or a foam.

Another object of the invention is a cosmetic use of a composition as defined herein, for combatting and/or reducing the signs of cutaneous ageing, such as the formation of wrinkles and/or fine lines, skin sagging, loss of firmness, loss of radiance and/or evenness of the complexion, and/or for reinforcing the skin barrier.

Another object of the invention is a kit comprising:
a composition as defined herein,
a washing composition, preferably a powder, a shampoo, a soap, a lotion, a solution, a solid, a scrubbing, a mousse, a foam, a syndet, a gel, a shower gel, a spray, a mist, a wax, a strip, or a woven or non-woven fabric, and
optionally an instruction guide.

A further object of the invention is a compound as defined herein or a pharmaceutical or veterinary composition as defined herein, for use for treating and/or preventing a skin, mucosa, eye cornea, or skin appendage disease or condition, preferably said skin, mucosa, eye cornea, or skin appendage disease or condition is chosen from lipodystrophy, keloid scars, acne, psoriasis, atopic dermatitis, actinic keratosis, rosacea, melasma, melanoma, Merker cell carcinoma, basal cell carcinoma, squamous cell carcinoma, scar treatments, wound healing, alopecia, vitiligo, urticaria (hives), cold sores, impetigo, eczema, rashes dermatitis, ichthyosis, warts, blisters, pruritus, gangrene, bruises, pustules, bacterial skin infections like leprosy, carbuncles, cellulitis, impetigo, fungal infections like Athlete's foot (intertrigo) and sporotrichosis, fungal nail infections, viral infection like herpes, sunburns, lice, scabies, pressure ulcer disinfection, and pressure ulcer healing.

In a particular embodiment, the composition as defined herein, or the composition for use as defined herein, further comprises a pharmaceutical or veterinary active ingredient. A further object of the invention is a material comprising a support and at least one compound as defined herein, said support being preferably a natural or synthetic polymeric support, a natural or synthetic fiber support, a stone, a metal, a plastic, a rubber, a glass support, or a paint.

### DETAILED DESCRIPTION OF THE INVENTION

### Definitions

As used herein, the expression a compound “derived from” a group or a group chosen in a list of groups means that the compound comprises a group or a group chosen in a list of groups including derivatives thereof, or preferably is a group or a group chosen in a list of groups including derivatives thereof. For instance, a photoprotective moiety A derived from a photoprotective moiety A chosen in the list of groups includes the photoprotective moiety of the list and any derivatives thereof comprising said photoprotective moiety.

As used herein, the term “aliphatic or aromatic carbocycle” refers to saturated or unsaturated, aliphatic or aromatic, mono-, bi- or tri-cyclic hydrocarbon, including fused, bridged, or spiro-connected carbocycles. In particular, the aliphatic or aromatic carbocycle may have 3 to 14 carbon atoms.

As used herein, the term “heterocycloalkyl” corresponds to an aliphatic, saturated or unsaturated, mono-, bi- or tri-cyclic hydrocarbon, further comprising at least one heteroatom within its cycle(s) such as O, N, or S. In particular, the heterocycloalkyl as defined herein may have 3 to 14 ring carbons and at least one (for instance, one, two three or four) heteroatom. It also includes fused, bridged, or spiro-connected heterocycloalkyl groups. Representative heterocycloalkyl groups include, but are not limited to tetraoxaspiro[5.5]undecanyl, 3-dioxolane, 7-oxabicyclo[2,2,1]heptanyl, benzo[1,3]dioxolyl, azetidinyl, oxetanyl, pyrazolinyl, pyranyl, thiomorpholinyl, pyrazolidinyl, piperidyl, piperazinyl, 1,4-dioxanyl, imidazolinyl, pyrrolinyl, pyrrolidinyl, piperidinyl, imidazolidinyl, morpholinyl, 1,4-dithianyl, pyrrolidinyl, oxozolinyl, oxazolidinyl, isoxazolinyl, isoxazolidinyl, thiazolinyl, thiazolidinyl, isothiazolinyl, isothiazolidinyl, dihydropyranyl, tetrahydropyranyl, tetrahydrofuranyl, and tetrahydrothiophenyl. A preferred heterocycloalkyl is tetraoxaspiro[5.5]undecanyl.

As used herein, the term “aliphatic or aromatic heterocycle” refers to an aliphatic or aromatic carbocycle as defined herein, further comprising at least one heteroatom, such as N, O, or S, within its cycle(s).

As used herein, the term “aryl” corresponds to a mono- or bi-cyclic aromatic hydrocarbon having from 6 to 14 carbon atoms. For instance, the term “aryl” includes phenyl, biphenyl, or naphthyl. In a preferred embodiment, the aryl is a phenyl.

As used herein, the term “heteroaryl” corresponds to an aromatic, mono- or poly-cyclic group comprising between 5 and 14 ring atoms and comprising at least one heteroatom such as nitrogen, oxygen or sulphur atom. Examples of such mono- and poly-cyclic heteroaryl group may be: pyridinyl, thiazolyl, thiophenyl, furanyl, pyrrolyl, pyrazolyl, imidazolyl, triazolyl, tetrazolyl, benzofuranyl, thianaphthalenyl, indolyl, indolinyl, quinolinyl, isoquinolinyl, benzimidazolyl, tetrahydroquinolinyl, tetrahydroisoquinolinyl, triazinyl, thianthrenyl, isobenzofuranyl, chromenyl, xanthenyl, phenoxanthinyl, isothiazolyl, isoxazolyl, pyrazinyl, pyridazinyl, indolizinyl, isoindolyl, indazolyl, purinyl, quinolizinyl, phtalazinyl, naphthyridinyl, quinoxalinyl, quinazolinyl, cinnolinyl, pteridinyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, indolinyl, isoindolinyl, oxazolidinyl, benzotriazolyl, benzoisoxazolyl, oxindolyl, benzoxazolinyl, benzothienyl, benzothiazolyl, isatinyl, dihydropyridyl, pyrimidinyl, s-triazinyl, oxazolyl, or thiofuranyl. A preferred heteroaryl is a triazinyl.

As used herein, the term “polyol” corresponds to an organic compound (preferably a linear or branched, cyclic or acyclic, aliphatic or aromatic hydrocarbon) comprising at least two alcohol groups (i.e. —OH), for instance two, three, four, five, six, seven, eight, nine or ten alcohol groups, and may comprise other functional groups, such as one or more ether bonds (—O—). Examples of polyols include, but are not limited to, ethylene glycol, propylene glycol, trimethylene glycol, butylene glycol, glycerol, erythritol, arabitol, ribitol, sorbitol, dulcitol, mannitol, volemitol, maltitol, isomaltitol, xylitol, or lactitol.

As used herein, the term “polyamine” corresponds to an organic compound (preferably a linear or branched, cyclic or acyclic, aliphatic or aromatic hydrocarbon) comprising at least two amine groups (i.e. —NH₂ or —NH—), for instance two, three, four, five, six, seven, eight, nine or ten amine groups, and may comprise other functional groups such as one or more oxo (═O) groups. Examples of polyamines include, but are not limited to, putrescine, cadaverine, spermidine, spermine, caldopentamine, agmatine, tris-(3-aminoethyl)amine, tris-(3-aminopropyl)amine, cyclen, triazacyclononane, tris(aminomethyl)ethane, or polyethyleneimine.

As used herein, the term “saccharide” corresponds to any organic compound comprising an aldehyde or ketone group and at least two hydroxy (—OH) group. The saccharide may be a monosaccharide or an oligo- or poly-saccharide (such as a disaccharide, trisaccharide, tetrasaccharide, pentasaccharide, or hexasaccharide). Examples of saccharide include, but are not limited to, glyceraldehyde, glucose, galactose, fructose, ribose, xylose, sucrose, lactose, maltose, isomaltose, trehalose, or raffinose.

As used herein, an amino-acid corresponds to any organic compound comprising an amino group (—NH₂) and a carboxylic acid group (—COOH). In particular, said amino-acid may be an alpha-amino-acid (i.e. having its NH₂ and COOH groups separated by one carbon atom), beta-amino-acid (i.e. having its NH₂ and COOH groups separated by two carbon atoms), or gamma-amino-acid (i.e. having its NH₂ and COOH groups separated by three carbon atoms). Said amino-acid may be a natural, non-natural, or a modified amino-acid. Examples of amino-acids include, but are not limited to, Glycine, Alanine, Valine, Leucine, Isoleucine, Proline, Serine, Threonine, Asparagine, Glutamine, Cysteine, Methionine, Phenylalanine, Tyrosine, Tryptophan, Aspartic acid, Glutamic acid, Histidine, Lysine, Arginine, Beta-alanine, or Cystine.

As used herein, a peptide corresponds to a homo- or hetero-oligomer or polymer of amino-acids as defined herein, linked together by peptide bonds (i.e. amide-type bonds). More particularly, the peptide may consist of from 2 to 200, preferably from 2 to 50, more preferably from 2 to 10 amino-acids as defined herein.

As used herein, the term “linear polymer” refers to a polymer which does not have any branches. The term “branched polymer” refers to a polymer having a linear chain substituted by primary branches, and optionally secondary branches. The term “hyperbranched polymer” refers to a polymer having randomly arranged primary and secondary branches. The term “dendrimer” (also referred to as “dendritic polymer” or “dendronized polymer”) refers to a repetitively branched polymer having a symmetric and organized tree-structure, in which branches originate from a common branch point.

The term “residue of a chain or a polymer” refers respectively to a chain or polymer deprived of one or more of its functional groups (such as —NH₂, —COOH, —OH), or atom(s) thereof (such as H or —OH).

The term “homopolymer” generally refers to a polymer that is composed of identical monomers. The term “copolymer” generally refers to a polymer that is composed of two or more different monomers. The copolymer can be of any form, such as random, block, or graft.

The copolymers can have any end-group.

The term “biodegradable linker (or compound)” refers to a linker (or a compound) that will degrade or erode under physical, chemical, and/or biological conditions to smaller units or chemical species that are capable of being metabolized, eliminated, or excreted by the subject.

The degradation time and speed are a function of composition and/or morphology. Degradation time can be from hours to weeks.

The term “biocompatible compound” refers to a compound that is, along with any metabolites or degradation products thereof, generally non-toxic to the recipient, and do not cause any significant adverse effects to the recipient. Generally speaking, biocompatible materials (or compounds are compounds which do not elicit a significant inflammatory or immune response when administered to a patient.

As used herein, the terms “prevent,” “preventing,” or “prevention,” refer to any reduction, no matter how slight, of a subject's predisposition or risk for developing a condition, disease, disorder or symptom thereof. For purposes of prevention, the subject is any subject, and preferably is a subject that is at risk for, or is predisposed to, developing a condition, disease, disorder. The term “prevention” includes either preventing the onset of a clinically evident condition, disease, disorder altogether or preventing the onset of a pre-clinically evident condition, disease, disorder in individuals at risk. This includes prophylactic treatment of subjects at risk of developing condition, disease, disorder.

As used herein, the terms “treat”, “treatment” or “treating” of a disease, disorder, or condition encompass alleviation of at least one symptom thereof, a reduction in the severity thereof, or the delay or inhibition of the progression thereof. Treatment need not mean that the disease, disorder, or condition is totally cured. A useful composition herein needs only to reduce the severity of a disease, disorder, or condition, reduce the severity of symptoms associated therewith, provide improvement to a patient or subject's quality of life, or delay or inhibit the onset of a disease, disorder, or condition.

“Effective amount” or “therapeutically effective amount”, as used herein, refers to an amount of drug or composition of the invention as disclosed herein effective to alleviate, delay onset of, or prevent one or more symptoms of a disease or disorder.

The present invention relates to multifilters, and more particularly to a compound comprising:
a plurality of (i.e. at least two) photoprotective moieties, linked to each other through a linker and/or to a radical (or equivalently “core”) through a linker, and
optionally at least one functional group, linked to said photoprotective moieties through a linker and/or to said core through a linker. The at least one functional group, when present, is able to render the compound of the invention adhesive, preferably bioadhesive.

The multifilters of the present invention exhibit an improved UV protection. As used herein, an improved photoprotection or an improved UV protection may refer to an enlarged UV protection scope, i.e. a protection against a broad UV spectrum. When applying to the skin, an improved photoprotection or an improved UV protection may include an enlarged skin UV protection scope, a more homogeneous covering on the skin, and/or a skin UV protection achieved with a reduced amount of multifilters. In a particular embodiment, an improved UV photoprotection or skin photoprotection or skin UV photoprotection means that the photoprotection is improved compared to current commercially available filters.

More specifically, a compound of the invention is represented by the following formula (I):

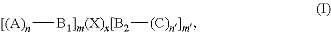

wherein:
X is a radical derived from an aryl, a heteroaryl, a polyol, a saccharide, an amino-acid, a peptide, or a polyamine;
each A represents independently a photoprotective moiety,
each B₁ and B₂ represents independently a linker,
C represents a functional group,
x is 0 or 1,
n is an integer from 1 to 12,
n′ is an integer from 1 to 2000,
m is an integer from 1 to 12,
m′ is an integer from 0 to 12, and
the number of photoprotective moieties A (m×n) is superior or equal to 2.

A compound of formula (I) according to the invention thus comprises:
at least one [(A)ₙ-B₁] moiety (m),
optionally a radical X (x), and
optionally at least one [B₂—(C)_{n′}] moiety (m′).

In a particular embodiment, m′ is 0. Accordingly, the compound of the invention does not comprise any [B₂—(C)_{n′}] moiety. Such compounds may comprise or not a radical X.

In another particular embodiment, m′ is an integer from 1 to 12. In such an embodiment, the compound of the invention comprises at least one [B₂—(C)_{n′}] moiety (m′). Such compounds of formula (I) in which m′ is an integer from 1 to 12, are also called herein “adhesive filters” or “bioadhesive filters”. Such compounds may comprise or not a radical X.

### Multifilters

According to the invention, a compound of formula (I) comprises m [(A)ₙ-B₁]moieties.

As used herein, m represents the number of [(A)ₙ-B₁]moieties in a compound of formula (I). According to the invention, m is an integer from 1 to 12. In particular embodiment, m is an integer from 1 to 6, preferably from 2 to 6, more preferably m is 2 to 4, even more preferably m is 2 or 3.

As used herein, n represents the number of photoprotective moieties A linked to each linker B₁. According to the invention, n is an integer from 1 to 12. In other words, each linker B₁ is independently linked to 1 to 12 photoprotective moieties A.

In particular embodiment, n is an integer from 1 to 6, preferably from 1 to 3, more preferably n is 1 or 2, and even more preferably n is 1.

As used herein, (m×n) represents the number of photoprotective moieties A in a compound of formula (I). According to the invention, (m×n) is superior or equal to 2. In other words, a compound of formula (I) according to the invention comprises at least two photoprotective moieties A, which may be identical to or different from each other.

The number of photoprotective moieties (m×n) is superior or equal to 2, which sets the following limitations: When n is 1, then m is an integer from 2 to 12. In other words, when a compound of formula (I) comprises one photoprotective moiety linked to each linker B₁, then said compound comprises 2 to 12 [(A)-B₁]moieties.

When m is 1, then n is an integer from 2 to 12. In other words, when a compound of formula (I) comprises one [(A)ₙ-B₁] moiety, then such moiety comprises 2 to 12 photoprotective moieties A each linked to the linker B₁.

In a particular embodiment, a compound of formula (I) is such that:
n is 1 and
m is an integer from 2 to 12, preferably from 2 to 6, more preferably from 2 to 4, even more preferably m is 2.

In another particular embodiment, a compound of formula (I) is such that:
m is 1 and
n is an integer from 2 to 12, preferably from 2 to 6, more preferably from 2 to 4, even more preferably n is 2.

As used herein, a “photoprotective moiety” refers to a photoprotective agent deprived of one or more atoms or atom groups, wherein the photoprotective activity of the photoprotective moiety is substantially similar to that of the photoprotective agent from which it derives. A photoprotective agent is typically an organic substance which can block, absorb, and/or increase (or “boost”) the absorption of all or part of a light, in particular, a light that can cause damages on the subject or material to be protected, such as Ultraviolet (“UV”) light. A photoprotective agent is typically a UV filter.

In a particular embodiment, each photoprotective moiety A is independently derived from a UV filter (or equivalently, a “UV absorber”). In particular, a UV filter may be a UVA filter, such as a UVA I or UVA II filter, a UV B filter, or a combination thereof. In a particular embodiment, at least one photoprotective moiety A is a UVA filter (such as a UVA I or UVA II filter) and at least one photoprotective moiety A is a UVB.

In another particular embodiment, each photoprotective moiety A is independently derived from a UV booster.

Examples of UV filters include, but are not limited to, bemotrizinol, diethylamino hydroxybenzoyl hexyl benzoate (DHHB, or “Uvinul© A Plus”), bisdisulizole disodium, meradimate, terephtalylidene dicamphor sulfonic acid (i.e. ecamsule), oxybenzone, sulisobenzone, iscotrizinol, octinoxate, octisalate, octyltriazone, padimate O, homosalate, amiloxate, octocrylene, PEG-25 PABA, ensulizole, trolamine salicylate, cinoxate, benzophenone-9, dioxybenzone, avobenzone, enzacamene, bisoctrizole, diethylhexyl naphthalate, diethylhexyl syringylidene, tetramethylhydroxy piperidinol, sodium benzotriazolyl butylphenol sulfonate, benzotriazole dodecyl p-cresol sulfonate, butyloctyl salicylate, bis(cyano butylacetate) anthracenediylidene, dimethylcapramide, ethyl hexyl methoxycrylene, ethyl hexyl triazone, 2,4,6-tri([1,1′-biphenyl]-4-yl)-1,3,5-triazine, 2-(4,6-diphenyl-1,3,5-triazin-2-yl)-5-[(hexyl)oxy]-phenol, 2,4-Bis(2,4-dimethylphenyl)-6-(2,4-dihydroxyphenyl)-1,3,5-triazine, or 4-(4,6-Bis((biphenyl-4-yl)-1,3,5-triazine-2-yl)-1,3-benzodiole.

In a particular embodiment, each photoprotective moiety A is independently derived from bemotrizinol, diethylamino hydroxybenzoyl hexyl benzoate, bisdisulizole disodium, meradimate, terephtalylidene dicamphor sulfonic acid, oxybenzone, sulisobenzone, iscotrizinol, octinoxate, octisalate, octyltriazone, padimate O, homosalate, amiloxate, octocrylene, PEG-25 PABA, ensulizole, trolamine salicylate, cinoxate, benzophenone-9, dioxybenzone, avobenzone, enzacamene, bisoctrizole, diethylhexyl naphthalate, diethylhexyl syringylidene, tetramethylhydroxy piperidinol, sodium benzotriazolyl butylphenol sulfonate, benzotriazole dodecyl p-cresol sulfonate, butyloctyl salicylate, bis(cyano butylacetate) anthracenediylidene, dimethylcapramide, ethyl hexyl methoxycrylene, ethyl hexyl triazone, 2,4,6-tri([, 1′-biphenyl]-4-yl)-1,3,5-triazine, 2-(4,6-diphenyl-1,3,5-triazin-2-yl)-5-[(hexyl)oxy]-phenol, 2,4-Bis(2,4-dimethylphenyl)-6-(2,4-dihydroxyphenyl)-1,3,5-triazine, or 4-(4,6-Bis((biphenyl-4-yl)-1,3,5-triazine-2-yl)-1,3-benzodiole.

In a more particular embodiment, each photoprotective moiety A is derived from diethylamino hydroxybenzoyl hexyl benzoate, butyloctyl salicylate, bemotrizinol, terephtalylidene dicamphor sulfonic acid, homosalate, octocrylene, ensulizole, 2-(4,6-diphenyl-1,3,5-triazin-2-yl)-5-[(hexyl)oxy]-phenol, octisalate, ethyl hexyl triazone, 2,4-Bis(2,4-dimethylphenyl)-6-(2,4-dihydroxyphenyl)-1,3,5-triazine, or 4-(4,6-Bis((biphenyl-4-yl)-1,3,5-triazine-2-yl)-1,3-benzodiole.

In a preferred embodiment, each photoprotective moiety A is derived from terephtalylidene dicamphor sulfonic acid, diethylamino hydroxybenzoyl hexyl benzoate, or butyloctyl salicylate.

In another preferred embodiment, each photoprotective moiety A is derived from bemotrizinol, diethylamino hydroxybenzoyl hexyl benzoate, terephtalylidene dicamphor sulfonic acid, homosalate, octocrylene, ensulizole, 2-(4,6-diphenyl-1,3,5-triazin-2-yl)-5-[(hexyl)oxy]-phenol, 2,4-Bis(2,4-dimethylphenyl)-6-(2,4-dihydroxyphenyl)-1,3,5-triazine, or 4-(4,6-Bis((biphenyl-4-yl)-1,3,5-triazine-2-yl)-1,3-benzodiole.

In a particular embodiment, each photoprotective moiety A is derived from a tris-aryltriazine. Examples of tris-aryltriazine include, but are not limited to, tris-aryltriazine compounds described in U.S. Pat. No. 8,106,108, such as 2,4,6-tri([1,1′-biphenyl]-4-yl)-1,3,5-triazine, 2-(4,6-diphenyl-1,3,5-triazin-2-yl)-5-[(hexyl)oxy]-phenol, 2,4-Bis(2,4-dimethylphenyl)-6-(2,4-dihydroxyphenyl)-1,3,5-triazine, or 4-(4,6-Bis((biphenyl-4-yl)-1,3,5-triazine-2-yl)-1,3-benzodiole. In another particular embodiment, each photoprotective moiety A is derived from a hydroxyphenyl benzotriazole. Examples of hydroxyphenyl benzotriazole include, but are not limited to, bisoctrizole, benzenepropanoic acid, 3-(2H-benzotriazol-2-yl)-5-(1,1-dimethylethyl)-4-hydroxy-, and C₇-C₉-branched or linear alkyl esters thereof.

According to the invention, the compound of formula (I) comprises at least one linker B₁. The linkers B₁ may be identical to or different from each other. As used herein, a “linker” refers to any organic chain having at least one carbon atom. Each linker B₁ may be independently linear, branched, hyperbranched, or dendritic. Each linker B₁ may be independently polymeric or not.

Preferably, each linker B₁ has independently from 1 to 250 carbon atoms, for instance from 1 to 50, from 1 to 12, from 1 to 10, from 1 to 8, or from 1 to 6 carbon atoms.

In a particular embodiment, the linker(s) B₁ is(are) biodegradable. The linker(s) B₁ may be derived from a compound of natural origin or bio-sourced.

In a particular embodiment, the linker(s) B₁ is(are) independently a saturated or unsaturated, linear or branched carbon chain, an aliphatic or aromatic carbocycle, an aliphatic or aromatic heterocycle, a linear polymer, a branched polymer, a hyperbranched polymer, a dendrimer, or a residue thereof (preferably a saturated or unsaturated, linear or branched carbon chain) and said linker optionally comprises at any one or both of its terminals a heteroatom independently chosen from —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—.

In such an embodiment, the linkers B₁ may be each independently interrupted by:
one or more heteroatomic groups chosen each independently from —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—, and/or
one or more heterocycloalkyls.

The polymer of said linear, branched, hyperbranched, or dendritic polymer may be a homopolymer or a copolymer. The polymer of said linear, branched, hyperbranched, or dendritic polymer may be natural, semi-synthetic, hemi-synthetic, or synthetic.

Examples of semi-synthetic, hemi-synthetic or synthetic polymers include, but are not limited to, poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyglycerol, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivatized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, hydroxypropyl methylcellulose acetate phthalate, carboxylethyl cellulose, cellulose triacetate, and cellulose sulfate sodium salt (jointly referred to herein as “synthetic celluloses”), polymers of acrylic acid, methacrylic acid or copolymers or derivatives thereof including esters, copolymers of acrylates and ammonium methacrylate, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof.

Examples of natural polymers include, but are not limited to, proteins such as albumin, collagen, gelatin and prolamines (e.g. zein), and polysaccharides such as alginate, dextran, chitosan, cellulose derivatives and polyhydroxyalkanoates (e.g. polyhydroxybutyrate) and microbial anatoxins.

In a particular embodiment, each linker B₁ is independently a linear saturated carbon chain having from 2 to 100 carbons, preferably from 2 to 20 carbons, more preferably from 2 to 10 carbons, for instance 2, 3, 4, 5, 6, 7, or 8 carbons,
said carbon chain optionally comprising at any one or both of its terminals a heteroatom independently chosen from —O—, —NH—, —O—C(O)—, —C(O)—O—, —C(O)—NH—, —NH—C(O)—, —NH—C(O)—O—, —O—C(O)—NH—, —SO₂—NH—, and —NH—SO₂— (preferably from —O— and —NH—), and said carbon chain being optionally interrupted by at least one (preferably one, two, or three) group chosen among —O—, tetraoxaspiro[5.5]undecanyl, —O—C(O)—, —C(O)—O—, —C(O)—NH—, and —NH—C(O)—.

In a more particular embodiment, each linker B₁ is a linear saturated carbon chain having from 2 to 10 carbons, for instance 2, 3, 4, 5, 6, 7, or 8 carbons,
said carbon chain optionally comprising at any one or both of its terminals a heteroatom independently chosen from —O—, —NH—, and
said carbon chain being optionally interrupted by at least one (preferably one, two, or three) group chosen among —O—, tetraoxaspiro[5.5]undecanyl, —O—C(O)—, and —C(O)—O— (preferably tetraoxaspiro[5.5]undecanyl).

In another particular embodiment, each linker B₁ is independently of formula (II):

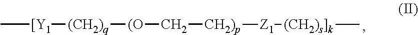

wherein:
Y₁ is selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—;
q is an integer from 0 to 35, preferably from 0 to 12, more preferably from 0 to 6;
with the proviso that, when Y₁ is —O—, q is different from 0;
p is an integer from 0 to 250, preferably from 0 to 50, more preferably from 0 to 12; and
p+q is different from 0; or
Y₁—(CH₂)_{q} may form a heterocycle selected in the group consisting of a pyrrolidinyl and piperidinyl and p is 0;
Z₁ is selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—;
s is an integer from 0 to 6; and
k is an integer from 1 to 4, preferably from 1 to 2.

More particularly, each linker B₁ may independently be of formula (II), wherein:
Y₁ is selected from a single bond, —O—, —NH—, —O—C(O)—, —C(O)—O—, —C(O)—NH—, —NH—C(O)—, —SO₂—NH—, and —NH—SO₂— (preferably from a single bond, —O— and —NH—);
q is an integer from 0 to 12;
   with the proviso that, when Y₁ is —O—, q is different from 0;
p is an integer from 0 to 3; and
p+q is different from 0;
Z₁ is selected from a single bond, —O—, —NH—, —O—C(O)—, —C(O)—O—, —C(O)—NH—, —NH—C(O)—, —SO₂—NH—, and —NH—SO₂— (preferably from a single bond, —O— and —NH—);
s is 0; and
k is 1.

Even more particularly, each linker B₁ may independently be of formula (II), wherein:
Y₁ and Z₁ are each independently selected from a single bond, —O—, and —NH—;
q is an integer from 0 to 12, more preferably from 0 to 6;
with the proviso that, when Y₁ is —O—, q is different from 0;
p is an integer from 0 to 12;
p+q is different from 0;
s is 0; and
k is i.

In another particular embodiment, each linker B₁ is independently of formula (III):

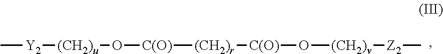

in which,
Y₂ and Z₂ are each independently selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—;
u and v are independently an integer from 1 to 50, preferably from 1 to 10, more preferably from 1 to 5, preferably u and v are independently 2, 3, or 4, and
r is an integer from 1 to 28, preferably from 1 to 12, more preferably r is 4.

More particularly, each linker B₁ may independently be of formula (III), wherein:
Y₂ and Z₂ are each independently a single bond, —O— or —NH—;
u and v are independently an integer from 1 to 10, more preferably from 1 to 5 (for instance, 2, 3 or 4), and
r is an integer from 1 to 28, preferably from 1 to 12, more preferably r is 4.

In another particular embodiment, each linker B₁ is independently of formula (IV):

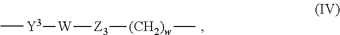

Y₃ and Z₃ are each independently selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—,
W is an aryl or a heteroaryl, preferably a phenyl; and
w is an integer from 1 to 150, preferably from 1 to 50, more preferably from 1 to 10.

Preferably, Y₃ and Z₃ are each independently a single bond, —O—, —NH—, —C(O)—, —O—C(O)—, —C(O)—O—, —C(O)—NH—, or —NH—C(O)—.

More preferably, Y₃ is a single bond or —NH—.

More preferably, Z₃ is —O—, —NH—, —C(O)—, —O—C(O)—, —C(O)—O—, —C(O)—NH—, or —NH—C(O)—.

Multi-Filters with m′ is 0

The present invention provides multifilters. According to a particular embodiment, compounds of formula (I) are such that m′ is 0.

In such embodiment, the compound of formula (I) comprises:
at least one [(A)ₙ-B₁] moiety (m) and
none [B₂—(C)_{n′}] moiety, nor none functional group C.

It is thus well understood that when m′ is 0, then n′ does not exist.

According to this embodiment, i.e. when m′ is 0, the at least two among the photoprotective moieties A are preferably different.

In a particular embodiment, m′ is 0 and x is 0. In such an embodiment, the compound of the invention does not comprise any [B₂—(C)_{n′}] moiety nor any radical X.

When m′ is 0 and x is 0, the m [(A)ₙ-B₁]moieties are thus typically linked to each other through the linker(s) B₁.

In a particular embodiment, the compound of the invention is of formula (I):

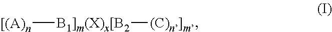

wherein:
X is a radical derived from an aryl, a heteroaryl, a polyol, a saccharide, an amino-acid, a peptide, or a polyamine;
each A represents independently a photoprotective moiety,
each B₁ and B₂ represents independently a linker,
C is a functional group,
x is 0,
n is an integer from 1 to 12,
n′ is an integer from 1 to 2000,
m is an integer from 1 to 12,
m′ is 0, and
the number of photoprotective moieties A (m×n) is superior or equal to 2,
preferably where at least two photoprotective moieties A are different.

In such embodiment, the compound of the invention can typically be represented by the following formula (I-1):

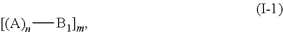

wherein:
A and B₁ are such as defined herein,
n is an integer from 1 to 12,
m is an integer from 1 to 12, and
the number of photoprotective moieties A (m×n) is superior or equal to 2,
preferably where at least two photoprotective moieties A are different.

In a more particular embodiment, the compound of formula (I) is such that:
x is 0,
n is an integer from 2 to 12,
m is 1, and
m′ is 0,
preferably provided that at least two photoprotective moieties A are different.

In such embodiment, the compound the invention can typically be represented by the following formula (I-1′):

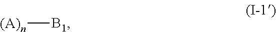

wherein:
A and B₁ are such as defined herein, and
n is an integer from 2 to 12,
preferably provided that at least two photoprotective moieties A are different.

The compound of formula (I-1′) comprises a linker B₁ and n photoprotective moieties A, each directly linked to the linker B₁, where at least two photoprotective moieties A are preferably different.

In a preferred embodiment, n is an integer from 2 to 6, more preferably from 2 to 4, for instance 2.

In an embodiment where n is 2, the compound of formula (I-1′) can be represented as follows:

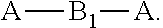

Each photoprotective moiety A is such as defined herein. Preferably, each photoprotective moiety A is independently derived from bemotrizinol, diethylamino hydroxybenzoyl hexyl benzoate, terephtalylidene dicamphor sulfonic acid, homosalate, octocrylene, ensulizole, 2-(4,6-diphenyl-1,3,5-triazin-2-yl)-5-[(hexyl)oxy]-phenol, 2,4-Bis(2,4-dimethylphenyl)-6-(2,4-dihydroxyphenyl)-1,3,5-triazine, or 4-(4,6-Bis((biphenyl-4-yl)-1,3,5-triazine-2-yl)-1,3-benzodiole.

Each linker B₁ is such as defined herein.

In a particular embodiment, each linker B₁ is independently a linear saturated carbon chain having from 2 to 100 carbons, preferably from 2 to 20 carbons, more preferably from 2 to 10 carbons, for instance 2, 3, 4, 5, 6, 7, or 8 carbons,
said carbon chain optionally comprising at any one or both of its terminals a heteroatom independently chosen from —O—, —NH—, —O—C(O)—, —C(O)—O—, —C(O)—NH—, —NH—C(O)—, —NH—C(O)—O—, —O—C(O)—NH—, —SO₂—NH—, and —NH—SO₂— (preferably from —O— and —NH—), and
said carbon chain being optionally interrupted by at least one (preferably one, two, or three) group chosen among —O—, tetraoxaspiro[5.5]undecanyl, —O—C(O)—, —C(O)—O—, —C(O)—NH—, and —NH—C(O)—.

In a more particular embodiment, each linker B₁ is a linear saturated carbon chain having from 2 to 10 carbons, for instance 2, 3, 4, 5, 6, 7, or 8 carbons, more preferably 6 or 8 carbons, said carbon chain optionally comprising at any one or both of its terminals a heteroatom independently chosen from —O—, —NH—, and said carbon chain being optionally interrupted by at least one (preferably one, two, or three) group chosen among —O—, tetraoxaspiro[5.5]undecanyl, —O—C(O)—, and —C(O)—O— (preferably tetraoxaspiro[5.5]undecanyl).

More particularly, each linker B₁ may independently be of formula (II), wherein:
Y₁ is selected from a single bond, —O—, —NH—, —O—C(O)—, —C(O)—O—, —C(O)—NH—, —NH—C(O)—, —SO₂—NH—, and —NH—SO₂— (preferably from a single bond, —O— and —NH—);
q is an integer from 0 to 12;
with the proviso that, when Y₁ is —O—, q is different from 0;
p is an integer from 0 to 3; and
p+q is different from 0;
Z₁ is selected from a single bond, —O—, —NH—, —O—C(O)—, —C(O)—O—, —C(O)—NH—, —NH—C(O)—, —SO₂—NH—, and —NH—SO₂— (preferably from a single bond, —O— and —NH—);
s is 0; and
k is 1.

Even more particularly, each linker B₁ may independently be of formula (II), wherein:
Y₁ and Z₁ are each independently selected from a single bond, —O—, and —NH—;
q is an integer from 0 to 12, more preferably from 0 to 6;
with the proviso that, when Y₁ is —O—, q is different from 0;
p is an integer from 0 to 12;
p+q is different from 0;
s is 0; and
k is 1.

In another particular embodiment, each linker B₁ may independently be of formula (III), wherein:
Y₂ and Z₂ are each independently a single bond, —O— or —NH—;
u and v are independently an integer from 1 to 10, more preferably from 1 to 5 (for instance, 2, 3 or 4), and
r is an integer from 1 to 28, preferably from 1 to 12, more preferably r is 4.

In a more particular embodiment, the linker B₁ is represented by one of the following formulae:

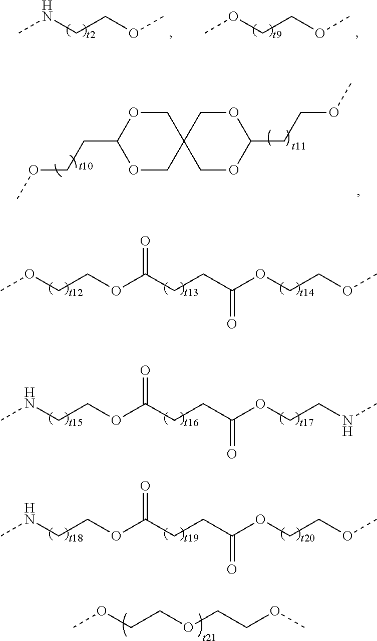

t2, t9, t10 and t11 are each independently an integer from 0 to 30, preferably from 1 to 12, more preferably from 2 to 10, still more preferably from 4 to 8, even still more preferably from 6 to 8, such as 6 or 8,
t12, t14, t15, t17, t18, and t20 are each independently an integer from 0 to 10, preferably from 0 to 5,
t13, t16, t19 are each independently an integer from 0 to 28, more preferably from 0 to 12, and
t21 is an integer from 0 to 250, preferably from 0 to 50, more preferably from 0 to 12.

In a particular embodiment, the compound of the invention is a compound of formula (I-4), wherein n is 2 and the linker B₁ is of formula (II). In such embodiment, the compound of the invention can typically be represented by the following formula (II-4):

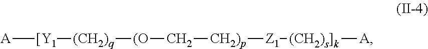

where A, n, Y₁, Z₁, q, p, s, and k are as defined above, including all the preferred and particular embodiments, with the proviso that the two photoprotective moieties A are different.

In a particular embodiment, the compound of the invention is a compound of formula (I-4), wherein n is 2 and the linker B₁ is of formula (III). In such embodiment, the compound of the invention can typically be represented by the following formula (III-4):

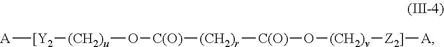

where A, n, Y₂, Z₂, u, v, and r are as defined herein, including all the preferred and particular embodiments, with the proviso that the two photoprotective moieties A are different.

In a preferred embodiment, the compound of the invention is selected from the group consisting of:
2-[(2-cyano-3,3-diphenyl-prop-2-enoyl)amino]ethyl 2-[4-(diethylamino)-2-hydroxy-benzoyl]benzoate;
8-[[2-[4-(diethylamino)-2-hydroxy-benzoyl]benzoyl]amino]octyl 2-cyano-3,3-diphenyl-prop-2-enoate;
8-[4-(4,6-diphenyl-1,3,5-triazin-2-yl)-3-hydroxy-phenoxy]octyl 2-hydroxybenzoate;
7-[4-[4,6-bis[4-(2-ethylhexoxy)-2-hydroxy-phenyl]-1,3,5-triazin-2-yl]phenoxy]heptyl 2-(4-amino-2-hydroxy-benzoyl)benzoate;
8-[4-(4,6-diphenyl-1,3,5-triazin-2-yl)-3-hydroxy-phenoxy]octyl 2-cyano-3,3-diphenyl-prop-2-enoate;
2-[2-[2-[4-[4,6-bis[4-(2-ethylhexoxy)-2-hydroxy-phenyl]-1,3,5-triazin-2-yl]phenoxy]ethoxy]ethoxy]ethyl 2-cyano-3,3-diphenyl-prop-2-enoate;
N-[7-[4-[4,6-bis[4-(2-ethylhexoxy)-2-hydroxy-phenyl]-1,3,5-triazin-2-yl]phenoxy]heptyl]-2-hydroxy-benzamide;
6-[[2-[4-(diethylamino)-2-hydroxy-benzoyl]benzoyl]amino]hexyl 2-hydroxybenzoate;
5-[5-(2-ethylhexoxy)-2-[4-[4-(2-ethylhexoxy)-2-hydroxy-phenyl]-6-(4-methoxyphenyl)-1,3,5-triazin-2-yl]phenoxy]pentyl 2-cyano-3,3-diphenyl-prop-2-enoate
N-[5-[4-[4,6-bis(2,4-dimethylphenyl)-1,3,5-triazin-2-yl]-3-hydroxy-phenoxy]pentyl]-2-[4-(diethylamino)-2-hydroxy-benzoyl]benzamide;
[(3Z)-3-[[4-[(Z)-[4-[7-[2-(2-hydroxy-4-methyl-benzoyl)benzoyl]oxyheptylsulfamoylmethyl]-7,7-dimethyl-3-oxo-norbornan-2-ylidene]methyl]phenyl]methylene]-7,7-dimethyl-2-oxo-norbornan-1-yl]methanesulfonic acid;
N-[5-[4-[4,6-bis(4-phenylphenyl)-1,3,5-triazin-2-yl]-3-hydroxy-phenoxy]pentyl]-2-[4-(diethylamino)-2-hydroxy-benzoyl]benzamide;
5-[(2-phenyl-3H-benzimidazol-5-yl)sulfonylamino]pentyl-2-[4-(diethylamino)-2-hydroxy-benzoyl]benzoate:
4-[9-[6-(2-cyano-3,3-diphenyl-prop-2-enoyl)oxyhexyl]-2,4,8,10-tetraoxaspiro[5.5]undecan-3-yl]butyl 2-[4-(diethylamino)-2-hydroxy-benzoyl]benzoate;
[(3Z)-3-[[4-[(Z)-[4-[3-[6-[2-(2-hydroxybenzoyl)oxyethoxy]-6-oxo-hexanoyl]oxypropylsulfamoylmethyl]-7,7-dimethyl-3-oxo-norbornan-2-ylidene]methyl]phenyl]methylene]-7,7-dimethyl-2-oxo-norbornan-1-yl]methanesulfonic acid;
O6-[3-(2-cyano-3,3-diphenyl-prop-2-enoyl)oxypropyl]01-[3-[2-[4-(diethylamino)-2-hydroxy-benzoyl]benzoyl]oxypropyl]hexanedioate;
[(3Z)-3-[[4-[(Z)-[4-[3-[6-[2-[(5-benzoyl-4-hydroxy-2-methoxy-phenyl)sulfonylamino]ethoxy]-6-oxo-hexanoyl]oxypropylsulfamoylmethyl]-7,7-dimethyl-3-oxo-norbornan-2-ylidene]methyl]phenyl]methylene]-7,7-dimethyl-2-oxo-norbornan-1-yl]methanesulfonic acid;
6-[[2-[4-(diethylamino)-2-hydroxy-benzoyl]benzoyl]amino]hexyl 2-cyano-3,3-diphenyl-prop-2-enoate; and
2-cyano-N-(8-(5-((2-ethylhexyl)oxy)-2-(4-(4-((2-ethylhexyl)oxy)-2-hydroxyphenyl)-6-(4-methoxyphenyl)-1,3,5-triazin-2-yl)phenoxy)octyl)-3,3-diphenylacrylamide.

In a more preferred embodiment, the compound of the invention is selected from the group consisting of:
8-[[2-[4-(diethylamino)-2-hydroxy-benzoyl]benzoyl]amino]octyl 2-cyano-3,3-diphenyl-prop-2-enoate;
6-[[2-[4-(diethylamino)-2-hydroxy-benzoyl]benzoyl]amino]hexyl 2-cyano-3,3-diphenyl-prop-2-enoate; and
2-cyano-N-(8-(5-((2-ethylhexyl)oxy)-2-(4-(4-((2-ethylhexyl)oxy)-2-hydroxyphenyl)-6-(4-methoxyphenyl)-1,3,5-triazin-2-yl)phenoxy)octyl)-3,3-diphenylacrylamide.

As used herein, 8-[[2-[4-(diethylamino)-2-hydroxy-benzoyl]benzoyl]amino]octyl 2-cyano-3,3-diphenyl-prop-2-enoate (or 8-(2-(4-(diethylamino)-2-hydroxybenzoyl)benzamido)octyl 2-cyano-3,3-diphenylacrylate) corresponds to compound #2 of the examples, also referenced as “OOD” (OCR-Octyl-DHHB with OCR is Octocrylene (2-ethylhexyl 2 cyano-3,3-diphenylacrylate and DHHB is diethylamino hydroxybenzoyl hexyl benzoate (Uvinul A plus).

As used herein, 6-[[2-[4-(diethylamino)-2-hydroxy-benzoyl]benzoyl]amino]hexyl 2-cyano-3,3-diphenyl-prop-2-enoate (or 6-(2-(4-(diethylamino)-2-hydroxybenzoyl)benzamido)hexyl 2-cyano-3,3-diphenylacrylate corresponds to compound #4 of the examples, also referenced as “OHD” (OCR-Hexyl-DHHB with OCR is Octocrylene (2-ethylhexyl 2 cyano-3,3-diphenylacrylate and DHHB is diethylamino hydroxybenzoyl hexyl benzoate (Uvinul A plus).

As used herein, 2-cyano-N-(8-(5-((2-ethylhexyl)oxy)-2-(4-(4-((2-ethylhexyl)oxy)-2-hydroxyphenyl)-6-(4-methoxyphenyl)-1,3,5-triazin-2-yl)phenoxy)octyl)-3,3-diphenylacrylamide corresponds to compound #5 of the examples, also referenced as “BOO” (BEMT-Octyl-OCR with OCR is Octocrylene (2-ethylhexyl 2 cyano-3,3-diphenylacrylate and BEMT is 6,6′-(6-(4-methoxyphenyl)-1,3,5-triazine-2,4-diyl)bis(3-((2-ethylhexyl)oxyphenol) (Tinosorb S or bemotrizinol).

In a particular embodiment, m′ is 0 and x is 1. In such an embodiment, the compound of the invention does not comprise any [B₂—(C)_{n′}] moiety but comprises a radical X.

As used herein, x represents the number of radical X in a compound of formula (I).

When m′ is 0 and x is 1, each of the m [(A)ₙ-B₁]moieties is thus typically linked to the radical X through their linker B₁.

In a particular embodiment, the compound of the invention is of formula (I):

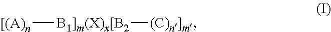

wherein:
X is a radical derived from an aryl, a heteroaryl, a polyol, a saccharide, an amino-acid, a peptide, or a polyamine;
each A represents independently a photoprotective moiety,
each B₁ and B₂ represents independently a linker,
C is a functional group,
x is 1,
n is an integer from 1 to 12,
n′ is an integer from 1 to 2000,
m is an integer from 1 to 12,
m′ is 0, and
the number of photoprotective moieties A (m×n) is superior or equal to 2,
preferably provided that at least two photoprotective moieties A are different.

In such embodiment, the compound of the invention can typically be represented by the following formula (I-2):

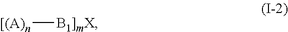

wherein:
X, A, and B₁ are such as defined herein, and
n is an integer from 1 to 12,
m is an integer from 1 to 12, and
the number of photoprotective moieties A (m×n) is superior or equal to 2,
preferably provided that at least two photoprotective moieties A are different.

In a more particular embodiment, the compound of formula (I) is such that:
x is 1,
n is 1,
m is an integer from 2 to 12, and
m′ is 0,
preferably, provided that at least two photoprotective moieties A are different.

In such embodiment, the compound of the invention can typically be represented by the following formula (I-2′):

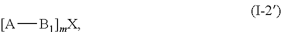

wherein:
X, A, and B₁ are such as defined herein, and
m is an integer from 2 to 12,
preferably, provided that at least two photoprotective moieties A are different.

The compound of formula (I-2′) comprises a radical X and m [A-B₁]moieties each linked to X through their linker B₁, where at least two photoprotective moieties A are preferably different.

In a preferred embodiment, m is an integer from 2 to 6, more preferably from 2 to 4, for instance 3.

X is a radical derived from an aryl, a heteroaryl, a polyol, a saccharide, an amino-acid, a peptide, or a polyamine.

In a particular embodiment, said radical X is derived from a phenyl, a triazinyl, a glycerol or a pentaerythritol. Preferably, X is a triazinyl.

Each photoprotective moiety A is such as defined herein. Preferably, each photoprotective moiety A is independently derived from bemotrizinol, diethylamino hydroxybenzoyl hexyl benzoate, terephtalylidene dicamphor sulfonic acid, homosalate, octocrylene, ensulizole, 2-(4,6-diphenyl-1,3,5-triazin-2-yl)-5-[(hexyl)oxy]-phenol, 2,4-Bis(2,4-dimethylphenyl)-6-(2,4-dihydroxyphenyl)-1,3,5-triazine, or 4-(4,6-Bis((biphenyl-4-yl)-1,3,5-triazine-2-yl)-1,3-benzodiole, more preferably from diethylamino hydroxybenzoyl hexyl benzoate or octocrylene.

Each linker B₁ is such as defined herein.

In a particular embodiment, each linker B₁ is independently a linear saturated carbon chain having from 2 to 10 carbons, for instance 2, 3, 4, 5, 6, 7, or 8 carbons, said carbon chain optionally comprising at any one or both of its terminals a heteroatom independently chosen from —O—, —NH—.

In a particular embodiment, each linker B₁ is independently of formula (II), wherein:
Y₁ and Z₁ are each independently selected from a single bond, —O—, and —NH—;
q is an integer from 1 to 12, more preferably from 1 to 8;
p is 0;
s is 0; and
k is 1.

In a more particular embodiment, each linker B₁ is independently represented by one of the following formulae:

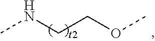

in which t2 is an integer from 0 to 30, preferably from 1 to 12.

In another particular embodiment, each moiety (A)ₙ-B₁ (or A-B₁) is independently represented by one of the following formulae:

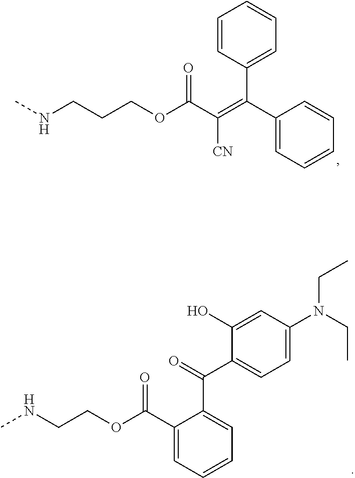

In a preferred embodiment, the compound of the invention is 2-[[4,6-bis[4-(2-cyano-3,3-diphenyl-prop-2-enoyl)oxybutylamino]-1,3,5-triazin-2-yl]amino]ethyl-2-(2-hydroxy-4-methyl-benzoyl)benzoate.

### Bioadhesive Multi-Filters

In another particular embodiment, the multifilters as defined herein have adhesive or bioadhesive properties.

The present invention thus provides adhesive or bioadhesive multifilters, and more particularly compounds of formula (I) as defined herein, wherein m′ is an integer from 1 to 12.

In such embodiment, the compound of formula (I) comprises:
at least one [(A)ₙ-B₁] moiety (m) and
at least one [B₂—(C)_{n′}] moiety (m′).

The at least one [B₂—(C)_{n′}] moiety (m′) makes the compounds of the invention adhesives or bioadhesives or improves their adhesion or bioadhesion.

As used herein, m′ represents the number of [B₂—(C)_{n′}] moieties in a compound of formula (I).

In particular embodiment, m′ is an integer from 1 to 6, preferably from 1 to 3, more preferably m is 1 or 2, and even more preferably m′ is 1.

As used herein, n′ represents the number functional groups C linked to each linker B₂, and is an integer from 1 to 2000. In other words, each linker B₂ is independently linked to 1 to 2000 functional groups C. For instance, when n′ is 2, each linker B₂ is linked to 2 functional groups C. In a particular embodiment, n′ is an integer from 100 to 1000, preferably from 150 to 500, and more preferably from 150 to 250. In another particular embodiment, n′ is 1 or 2, preferably 1.

The total number of functional groups C in a compound of formula (I) is defined by (m′×n′).

In a particular embodiment, m′ is an integer from 1 to 12, and x is 0. In such an embodiment, the compound of the invention comprises at least one [B₂—(C)_{n′}] moiety but does not comprise any radical X.

When m′ is an integer from 1 to 12, and x is 0, the m [(A)ₙ-B₁] moieties are thus typically linked to each other through their linker B₁ and/or linked to the at least one [B₂—(C)_{n′}] moiety through their linker B₁, and reciprocally, the at least one [B₂—(C)_{n′}] moiety is linked to each other through their linker B₂ and/or linked to the [(A)ₙ-B₁]moieties through their linker B₂.

In a particular embodiment, m′ is an integer from 1 to 12, and x is 1. In such an embodiment, the compound of the invention comprises at least one [B₂—(C)_{n′}] moiety and a radical X.

When m′ is an integer from 1 to 12, and x is 1, each of the m [(A)ₙ-B₁]moieties is typically linked to the radical X through their linker B₁ and each of the m′ [B₂—(C)_{n′}] moieties are typically linked to the radical X through their linker B₂.

In a particular embodiment, the compound of the invention is of formula (I):

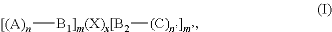

wherein:
X is a radical derived from an aryl, a heteroaryl, a polyol, a saccharide, an amino-acid, a peptide, or a polyamine;
each A represents independently a photoprotective moiety,
each B₁ and B₂ represents independently a linker,
C is a functional group,
x is 1,
n is an integer from 1 to 12,
n′ is an integer from 1 to 2000,
m is an integer from 1 to 12,
m′ is an integer from 1 to 12, and
the number of photoprotective moieties A (m×n) is superior or equal to 2.

In such embodiment, the compound of the invention can typically be represented by the following formula (I-3):

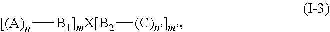

wherein:
X, A, B₁, B₂, and C are such as defined herein, and
n is an integer from 1 to 12,
n′ is an integer from 1 to 2000,
m is an integer from 1 to 12,
m′ is an integer from 1 to 12, and
the number of photoprotective moieties A (m×n) is superior or equal to 2.

In a more particular embodiment, the compound of formula (I) is such that:
x is 1,
n is 1,
m is an integer from 2 to 12, and
m′ is an integer from 1 to 12.

In such embodiment, the compound of the invention can typically be represented by the following formula (I-3′):

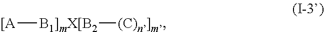

wherein:
X, A, B₁, B₂, and C are such as defined herein,
n′ is an integer from 1 to 2000,
m is an integer from 2 to 12, and
m′ is an integer from 1 to 12.

The compound of formula (I-3′) comprises a radical X, m [A-B₁]moieties each linked to X through their linker B₁ and m′ [B₂—(C)_{n′}]moieties each linked to X through their linker B₂.

In a preferred embodiment, n′ is 1.

In another preferred embodiment, m is an integer from 2 to 6, more preferably from 2 to 4, even more preferably m is 2.

In another preferred embodiment, m′ is 1 or 2, more preferably m′ is 1.

The radical X is such as above defined. Preferably, X is a triazinyl.

Each photoprotective moiety A is such as defined herein. Preferably, each photoprotective moiety A is independently derived from terephtalylidene dicamphor sulfonic acid, diethylamino hydroxybenzoyl hexyl benzoate, or butyloctyl salicylate.

According to the invention, C represents a functional group. The total number of functional groups C in a compound of formula (I) is defined by (m′×n′). When the total number of functional groups is at least two, the functional groups may be identical to or different from each other.

As used herein, a “functional group” is a group that is able to react with or bound to any support of any material, any tissues, cells, intracellular or extracellular materials. Such functional group(s) C, when present, makes the compound of formula (I), adhesive or bioadhesive.

In a particular embodiment, each functional group C is independently chosen from an aldehyde, an acetal, thiocetal, a thiol, a maleimide, a 5-methylene pyrrolone, a 3-bromo-5-methylene pyrrolone, a 5-hydroxy-pyrrolone, a 3,3-dimethylacrylic acid, a Mickael acceptor, a vinylsulfone, a disulfanylpyridine, a sulfonylaziridine, a vinylheteroarene, a vinylpyridine, a vinylpyrimidine, a vinyltriazine, a vinyltetrazine, an epoxide, a haloacetyl, an isocyanate, an isothiocyanate, a N-hydroxysuccinimide ester, a N-hydroxysulfosuccinimide ester, a hydroxy, an amino, an ammonium, a guanidinium, an imidocarbonate, a carboxylic acid, a carboxylic ester, an anhydride, a sulfonic acid, folic acid, biotin, streptavidin, avidin, antibodies, and single chain antibodies or fragments thereof. In a more particular embodiment, C further includes any derivatives of such functional groups as above detailed.

For instance, the term “maleimide” (Mal) refers to the maleimide group and any derivative thereof. In particular, the maleimide (Mal) includes the two following groups of formula (Mal 1) and (Mal 2):

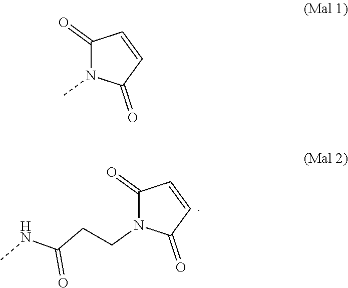

As used in the formulae disclosed in the present application, the symbol “ [symbol] ” in a formula represents the bond by which a moiety of said formula is attached to the remainder of the molecule. For instance, said symbol represented above in (Mal 1) or (Mal 2), represents the bond by which the moiety is attached to the linker B₂.

Preferably, each functional group C is independently chosen from a maleimide, a 5-methylene pyrrolone and derivatives thereof.

In a further particular embodiment, C represents a native functional group.

As used herein, a “native functional group” of a polymer is a functional group which is intrinsically present in the structure of the polymer and thus has not been converted into another functional group. For instance, hydroxy groups are native functional groups of cellulose, and carboxylic acids (or carboxylate) are native functional groups of a polymethacrylic acid.

In a further particular embodiment, C represents a modified functional group.

As used herein, a “modified functional group” of a polymer is a functional group that results from the conversion of a native functional group as defined herein into another functional group, for instance, an aldehyde group resulting from oxidation of a hydroxy group of cellulose or a biotin linked to a hydroxy group of cellulose.

Preferably, each functional group C is independently chosen from a maleimide, a 5-methylene pyrrolone and derivatives thereof.

The linkers B₁ and B₂ may be identical or different. The linkers B₁ may be identical to or different from each other.

The linker(s) B₁ is(are) such as defined herein. In a particular embodiment, each linker B₁ is independently a linear saturated carbon chain having from 2 to 10 carbons, for instance 2, 3, 4, 5, 6, 7, or 8 carbons,
said carbon chain optionally comprising at any one or both of its terminals a heteroatom independently chosen from —O— and —NH— and
said carbon chain being optionally interrupted by at least one (preferably one, two, or three) group chosen among —O—, —C(O)—NH—, and —NH—C(O)—.

In another particular embodiment, each linker B₁ is of formula (II), wherein:
Y₁ and Y₂ are each independently selected from a single bond, —O— and —NH— (preferably —NH—
q is an integer from 0 to 12;
with the proviso that, when Y₁ is —O—, q is different from 0;
p is an integer from 0 to 3;
p+q is different from 0;
s is 0; and
k is 1.

In a more particular embodiment, each linker B₁ is independently represented by one of the following formulae:

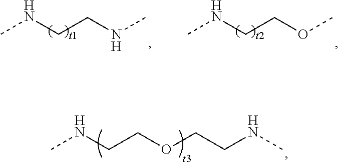

in which:
t1 and t2 are each independently an integer from 0 to 30, preferably from 1 to 12, and
t3 is an integer from 0 to 250, preferably from 0 to 50, more preferably from 0 to 12.

In another particular embodiment, each moiety (A)ₙ-B₁ (or A-B₁) is independently represented by one of the following formulae:

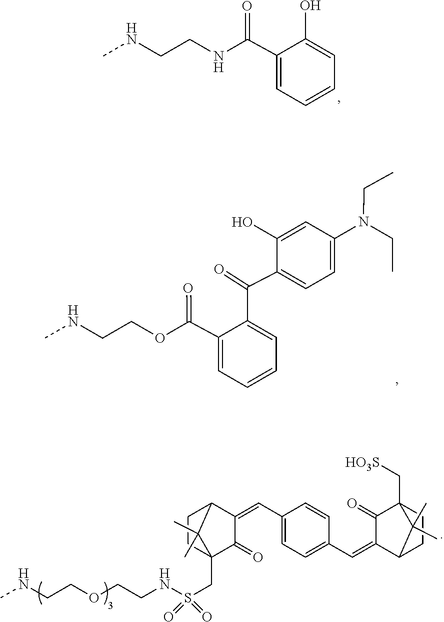

Each linker B₂ may be independently linear, branched, hyperbranched, or dendritic. Each linker B₂ may be independently polymeric or not. Preferably, each linker B₂ has independently from 1 to 250 carbon atoms, for instance from 1 to 50, from 1 to 12, from 1 to 10, from 1 to 8 or from 1 to 6 carbon atoms.

In a particular embodiment, the linker(s) B₂ is(are) biodegradable. The linker(s) B₂ may be derived from a compound of natural origin or bio-sourced. When m′ is at least 2, the linkers B₂ may be identical to or different from each other.

In a particular embodiment, each linker B₂ is independently a saturated or unsaturated, linear or branched carbon chain, an aliphatic or aromatic carbocycle, an aliphatic or aromatic heterocycle, a linear polymer, a branched polymer, a hyperbranched polymer, a dendrimer, or a residue thereof (preferably a saturated or unsaturated, linear or branched carbon chain) and said linker optionally comprises at any one or both of its terminals a heteroatom independently chosen from —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—.

In such an embodiment, the linkers B₂ may be each independently interrupted by:
one or more heteroatomic groups chosen each independently from —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—, and/or
one or more heterocycloalkyls.

In a more particular embodiment, each linker B₂ is independently a linear saturated carbon chain having from 2 to 100 carbons, preferably from 2 to 20 carbons, more preferably from 2 to 10 carbons, for instance 2, 3, 4, 5, 6, 7, or 8 carbons,
said carbon chain optionally comprising at any one or both of its terminals a heteroatom independently chosen from —O—, —NH—, —O—C(O)—, —C(O)—O—, —C(O)—NH—, —NH—C(O)—, —NH—C(O)—O—, —O—C(O)—NH—, —SO₂—NH—, and —NH—SO₂— (preferably from —O— and —NH—), and
said carbon chain being optionally interrupted by at least one (preferably one, two, or three) group chosen among —O—, tetraoxaspiro[5.5]undecanyl, —O—C(O)—, —C(O)—O—, —C(O)—NH—, and —NH—C(O)—.

In an even more particular embodiment, each linker B₂ is a linear saturated carbon chain having from 2 to 10 carbons, for instance 2, 3, 4, 5, 6, 7, or 8 carbons,
said carbon chain optionally comprising at any one or both of its terminals a heteroatom independently chosen from —O—, —NH—, and
said carbon chain being optionally interrupted by at least one (preferably one, two, or three) group chosen among —O—, tetraoxaspiro[5.5]undecanyl, —O—C(O)—, and —C(O)—O— (preferably tetraoxaspiro[5.5]undecanyl).

In an even more particular embodiment, each linker B₂ is independently a linear saturated carbon chain having from 2 to 10 carbons, for instance 2, 3, 4, 5, 6, 7, or 8 carbons,
said carbon chain optionally comprising at any one or both of its terminals a heteroatom independently chosen from —O— and —NH— and
said carbon chain being optionally interrupted by at least one (preferably one, two, or three) group chosen among —O—, —C(O)—NH—, and —NH—C(O)—.

In another particular embodiment, each linker B₂ is independently of formula (II):

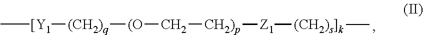

wherein:
Y₁ is selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—;
q is an integer from 0 to 35, preferably from 0 to 12, more preferably from 0 to 6;
   with the proviso that, when Y₁ is —O—, q is different from 0;
p is an integer from 0 to 250, preferably from 0 to 50, more preferably from 0 to 12; and
p+q is different from 0; or
Y₁—(CH₂)_{q} may form a heterocycle selected in the group consisting of a pyrrolidinyl and piperidinyl and p is 0;
Z₁ is selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—;
s is an integer from 0 to 6; and
k is an integer from 1 to 4, preferably from 1 to 2.

More particularly, each linker B₂ may independently be of formula (II), wherein:
Y₁ is —O—;
q is an integer from 1 to 12;
p is 0;
Z₁ is selected from a single bond, —NH—, —C(O)—NH—, and —NH—C(O)—;
s is an integer from 0 to 2; and
k is 1.

In another particular embodiment, each linker B₂ is independently of formula (III):

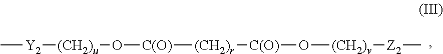

in which,
Y₂ and Z₂ are each independently selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—;
u and v are independently an integer from 1 to 50, preferably from 1 to 10, more preferably from 1 to 5, preferably u and v are independently 2, 3, or 4, and
r is an integer from 1 to 28, preferably from 1 to 12, more preferably r is 4.

In another particular embodiment, each linker B₂ is independently of formula (IV):

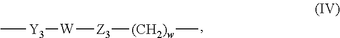

Y₃ and Z₃ are each independently selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO₂—NH—, and —NH—SO₂—,
W is an aryl or a heteroaryl, preferably a phenyl; and
w is an integer from 1 to 150, preferably from 1 to 50, more preferably from 1 to 10.

Preferably, Y₃ and Z₃ are each independently a single bond, —O—, —NH—, —C(O)—, —O—C(O)—, —C(O)—O—, —C(O)—NH—, or —NH—C(O)—.

More preferably, Y₃ is a single bond or —NH—.

More preferably, Z₃ is —O—, —NH—, —C(O)—, —O—C(O)—, —C(O)—O—, —C(O)—NH—, or —NH—C(O)—.

In another particular embodiment, each linker B₂ is independently of formula (IV), wherein:
Y₃ and Z₃ are each independently selected from a single bond, —O— and —NH—,
W is a phenyl; and
w is an integer from 1 to 10, preferably from 2 to 6.

In a more particular embodiment, each linker B₂ is independently represented by one of the following formulae:

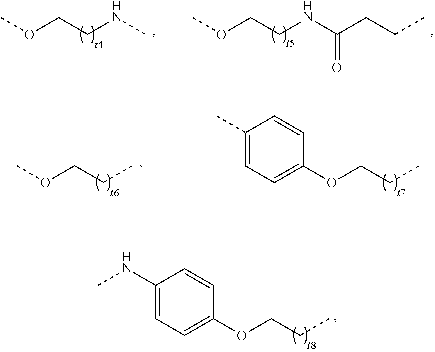

in which t4, t5, t6, t7, and t8 are each independently an integer from 0 to 30, preferably from 0 to 12.

In another particular embodiment, each moiety —B₂—(C)_{n′} is independently represented by one of the following formulae:

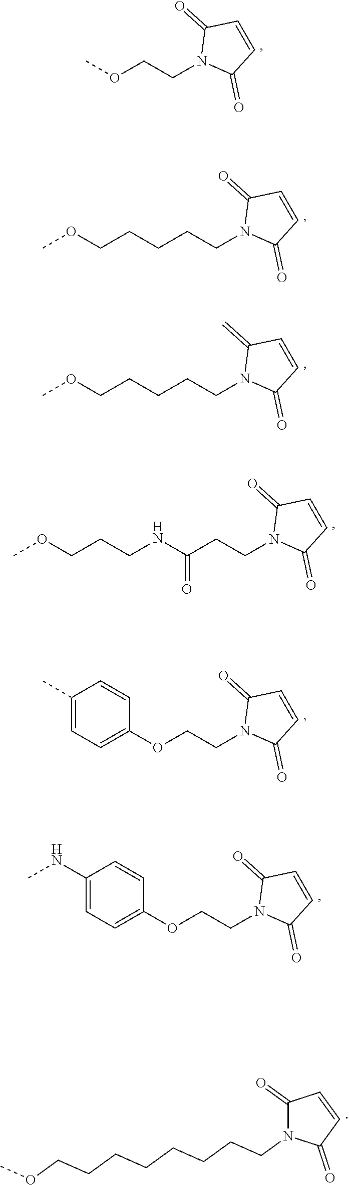

In a particular embodiment, the compound of the invention is a compound of formula (I-1) wherein X is a triazinyl, more particularly a 1,3,5-triazinyl.

In such embodiment the compound of the invention can be represented by the following formula (I-4):

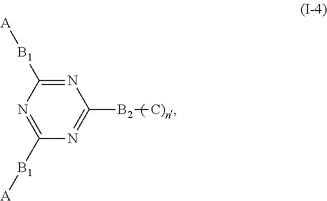

wherein A, each B₁, B₂, C, and n′ are such as defined above, including all the preferred and particular embodiments. Preferably, n′ is 1.

In a preferred embodiment, the compound of the invention is one of the following compounds:
2-[[4-[2-(2,5-dioxopyrrol-1-yl)ethoxy]-6-[2-[(2-hydroxybenzoyl)amino]ethylamino]-1,3,5-triazin-2-yl]amino]ethyl 2-[4-(diethylamino)-2-hydroxy-benzoyl]benzoate;
N-[2-[[4-[5-(2,5-dioxopyrrol-1-yl)pentoxy]-6-[2-[2-[2-[2-[[rac-(3Z,4R)-7,7-dimethyl-2-oxo-3-[[4-[rac-(Z)-[rac-(1R)-4-ethyl-7,7-dimethyl-3-oxo-norbornan-2-ylidene]methyl]phenyl]methylene]norbornan-1-yl]methylsulfonylamino]ethoxy]ethoxy]ethoxy]ethylamino]-1,3,5-triazin-2-yl]amino]ethyl]-2-hydroxy-benzamide;
2-hydroxy-N-[2-[[4-[5-(2-methylene-5-oxo-pyrrol-1-yl)pentoxy]-6-[2-[2-[2-[2-[[rac-(3Z,4R)-7,7-dimethyl-2-oxo-3-[[4-[rac-(Z)-[rac-(1R)-4-ethyl-7,7-dimethyl-3-oxo-norbornan-2-ylidene]methyl]phenyl]methylene]norbornan-1-yl]methylsulfonylamino]ethoxy]ethoxy]ethoxy]ethylamino]-1,3,5-triazin-2-yl]amino]ethyl]benzamide;
2-[[4-[4-[2-(2,5-dioxopyrrol-1-yl)ethoxy]anilino]-6-[2-[(2-hydroxybenzoyl)amino]ethyl amino]-1,3,5-triazin-2-yl]amino]ethyl 2-[4-(diethylamino)-2-hydroxy-benzoyl]benzoate;
2-[[4-[4-[2-(2,5-dioxopyrrol-1-yl)ethoxy]phenyl]-6-[2-[(2-hydroxybenzoyl)amino]ethyl amino]-1,3,5-triazin-2-yl]amino]ethyl 2-[4-(diethylamino)-2-hydroxy-benzoyl]benzoate; and
2-cyano-N-(8-(2-(4-(2-((8-(2,5-dioxo-2,5-dihydro-1H-pyrrol-1-yl)octyl)oxy)-4-((2-ethylhexyl)oxy)phenyl)-6-(4-methoxyphenyl)-1,3,5-triazin-2-yl)-5-((2-ethylhexyl)oxy)phenoxy)octyl)-3,3-diphenylacrylamide.

As used herein, 2-cyano-N-(8-(2-(4-(2-((8-(2,5-dioxo-2,5-dihydro-1H-pyrrol-1-yl)octyl)oxy)-4-((2-ethylhexyl)oxy)phenyl)-6-(4-methoxyphenyl)-1,3,5-triazin-2-yl)-5-((2-ethylhexyl)oxy)phenoxy)octyl)-3,3-diphenylacrylamide, corresponds to compound #5 of the examples, also referenced as “BOO-OM” (BEMT-Octyl-OCR-Octyl-Mal with OCR is Octocrylene (2-ethylhexyl 2 cyano-3,3-diphenylacrylate, BEMT is 6,6′-(6-(4-methoxyphenyl)-1,3,5-triazine-2,4-diyl)bis(3-((2-ethylhexyl)oxyphenol) (Tinosorb S or bemotrizinol), and Mal is maleimide.

As used herein, the expression “adhesive compound” denotes a compound that is able to adhere, through one or more functional groups (e.g. functional groups C as defined herein), when present, to any support, said support being biological, organic, and/or inorganic. More specifically, said compound adheres through one or more of its functional groups (e.g. functional groups C as defined herein), when present, which are able to react with reactive groups or entities of said support.

A “bioadhesive compound” refers to an adhesive compound as defined above, wherein the support is biological. Examples of biological supports include but are not limited to tissues (e.g. a skin), cells (e.g. chondrocytes, osteoblasts, fibroblasts, blood cells, plasmocytes), intracellular or extracellular materials (e.g. proteins, glycoproteins, collagen, elastin, glycosaminoglycans, proteoglycans).

Reactive groups that can be found on said supports, and more particularly on said biological supports, include, but are not limited to, amine, ammonium, guanidinium, thiol, carboxylic acid, and carboxylate.

Said functional groups C may advantageously react selectively with a particular reactive group found on said support.

For instance:
maleimide, thiol, 3,3-dimethylacrylic acid, a 5-methylene pyrrolone, Mickael acceptors, sulfonylazeridine, vinylsulfone, isocyanate, or thiocyanate can typically be selective to thiol groups;
carboxylic acid, aldehyde, acetal, esters, NHS esters, sulfo-NHS esters, or anhydride can typically be selective to amine groups;
carboxylate can typically be selective to ammonium;
amine can typically be selective to carboxylic acid; and
ammonium and guanidinium can typically be selective to carboxylate.

Reaction of a functional group C of a compound of formula (I) with a reactive group creates a bond such as amide, disulfide, thioether, thiocarbamate, imine, or ionic pair —NH₃⁺, —OOC—. The bound created between a functional group C of a compound of formula (I) and a reactive group of a support may be covalent or ionic. Said bound is advantageously reversible. Said bound may be cleaved by use of a cleaving material selected from chemical and physical agents (e.g. protein, peptide (e.g. glutathione), amino acid, enzyme (e.g. cathepsin B), thiol (e.g. 2-mercaptoethanol, N-acetyl cysteine), dithiol (e.g. dithiothreitol), pH-modifier, acid, base, solvent, and/or woven or non-woven tissue. The skilled artisan is able to select an appropriate cleaving material depending on the nature of the bound and/or the composition.

In a particular embodiment, a compound of formula (I) (or of formula (I-1), (I-1′), (I-2), (I-2′), (I-3), (I-3′) or (I-4)) is biocompatible.

In another particular embodiment, a compound of formula (I) (of formula (I-1), (I-1′), (I-2), (I-2′), (I-3), (I-3′) or (I-4)) is biodegradable. A compound of formula (I) (of formula (I-1), (I-1′), (I-2), (I-2′), (I-3), (I-3′) or (I-4)) may be derived from compounds of natural origin (or bio-sourced) or prepared from compounds of natural origin (or bio-sourced).

Composition Another object of the invention is a composition comprising at least one compound of formula (I) (or of formula (I-1), (I-1′), (I-2), (I-2′), (I-3), (I-3′) or (I-4)) as defined herein and at least one excipient.

In a particular embodiment, the composition comprises from 0.01 wt % to 99 wt % of compound of formula (I) (or of formula (I-1), (I-1′), (I-2), (I-2′), (I-3), (I-3′) or (I-4)), preferably from 0.01 wt % to 90 wt %, more preferably from 1 wt % to 70 wt %, even more preferably from 5 wt % to 50 wt %, relative to the total weight of the composition.

The composition of the invention may comprise a plurality of compounds of formula (I), of formula (I-1), (I-1′), (I-2), (I-2′), (I-3), (I-3′) and/or (I-4). In particular, said composition may comprise a plurality of compounds of formula (I) (or (I-3), or (I-3′)) having different ratios of the number of functional groups C (i.e. m′×n′) to the number of photoprotective moieties A (i.e. m×n). For instance, said composition may comprise:
at least one compound of formula (I) (or (I-3), or (I-3′)) having one functional group C and two photoprotective moieties A (i.e. ratio (m′×n′)/(m×n)=1/2); and
at least one compound of formula (I) (or (I-3), or (I-3′)) having two functional groups C and two photoprotective moieties A (i.e. ratio (m′×n′)/(m×n)=2/2).

The composition according to the invention may in particular be in the form of a suspension, a cream, a spray, an aerosol, a butter, a stick, a gel, an ointment, a lotion, a solution, a solid, an emulsion, a micro-emulsion, an oil, a lyophilizate, a milk, a powder, a paste, a wax, a mousse, a patch, a film, a micelle, a liposome, or a foam. The composition can be prepared according to processes known to the skilled artisan.

The composition of the invention may comprise a solvent or a dispersion medium comprising, for instance, water, ethanol, one or more polyols (e.g., glycerol, propylene glycol, and liquid polyethylene glycol), oils, such as vegetable oils (e.g., peanut oil, corn oil, sesame oil, etc.), and combinations thereof.

Examples of excipients include, but are not limited to, surfactants, dispersants, emulsifiers, pH modifying agents, pH-buffers, viscosity modifying agents, preservatives, polymerizers, pigments, colorants, stabilizing agents, glidants, diluents, binders, water-soluble polymers, lubricants, disintegrators, swelling agents, fillers, stabilizers, antioxidants, emulsifiers, emollients, penetration enhancers, propellants, gas, depigmenting agents, film forming agents, gelling agents, moisturizing agents, colorants, fragrance ingredients, exfoliants, solubilizers, solvents, binding agents, bulking agents, humectants, cleansing agents, elastomers, astringents, masking agents, anti-static agents, protectants, denaturants, absorbents, anti-caking agents, matting agents, structuring agents, oxidative agents, reducing agents, superfatting agents, active boosters, and combinations thereof.

Examples of additional agents which may be comprised in the composition include, but are not limited to, desquaming agents, whitening agents, tensing effect agents soothing agents, anti-irritant agents, sebo-regulating agents, wound healing agents, anti-inflammatory agents, anti-acne agents, anti-glycation agents, slimming agents, self-tanning agents, anti-aging agents, anti-wrinkle agents,

Surfactants that can be used in the composition may be anionic, cationic, amphoteric or nonionic. Examples of anionic surfactants include, but are not limited to, carboxylate, sulfonate and sulfate ions-containing surfactants, such as sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; sulfated castor oil, propylene glycol, lecithin, capric/caprylic triglycerides, PEG-12 oleate (FANCOL® HS3 US®), and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, polyoxyethylene hydrogenated tallow amide, but also emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone, cetyl palmitate. Examples of amphoteric surfactants include sodium N-dodecyl-.beta.-alanine, sodium N-lauryl-.beta.-iminodipropionate, myristoamphoacetate, lauryl betaine and lauryl sulfobetaine.

Examples of preservatives, include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, perillic acid and thimerosal.

Examples of water-soluble polymers include, but are not limited to, polyvinylpyrrolidone, dextran, carboxymethylcellulose, and polyethylene glycol.

Suitable stabilizers include, but are not limited to, butylated hydroxytoluene (BHT), ascorbic acid, its salts and esters, Vitamin E, tocopherol and its salts, sulfites such as sodium metabisulphite, cysteine and its derivatives, citric acid, propyl gallate, and butylated hydroxyanisole (BHA).

An example of pH-buffer that can be used in the composition is triethanolamine.

Examples of emollients include, but are not limited to, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof.

Examples of emulsifiers include, but are not limited to, acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum, PEG-100 Stearate/Glyceryl stearate (Arlacel 165®), decyglucoside (Plantaren 2000®), laurylglucoside (Plantaren1200®), Cetearyl Glucoside, Cetearyl Alcohol (Emulgade PL68/50®), and combinations thereof.

Examples of penetration enhancers include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols).

Examples of propellant agents include, but are not limited to, dichlorofluoromethane, difluoroethane, isobutane, n-butane, propane, dichlorofluoromethane, nitrogen, carbon dioxide.

Examples of desquaming agents include, but are not limited to, beta hydroxyacids, alpha hydroxy acids, urea, cinnamic acid, Saphora japonica extract, proteases like trypsine.

Examples of depigmenting agents include, but are not limited to, vitamin C and its derivatives, ferulic acid, resorcinol, alpha and beta arbutin.

Examples of anti-glycation agents include, but are not limited to, black tea extract and Vaccinium myrtillus extract.

Examples of slimming agents include, but are not limited to, caffeine, tea extracts, Hedera helix extracts, and theobromine.

Examples of soothing agents and anti-irritation agents include, but are not limited to, caffeine, vitamins E, C, B5, B3, glycyrrhetic acid, a salt or a derivative thereof.

Examples of sebo-regulating agents include, but are not limited to, zinc salts such as zinc gluconate or zinc pidolate, vitamin B₆, selenium chloride, and benzoyl peroxide.

Examples of wound healing agents include, but are not limited to, arginine, hydroxyproline, chitosan and derivatives, propolis extracts, folic acid, and chitosan.

An example of self-tanning agent includes, but is not limited to, erythrulose.

Examples of anti-aging agents include, but are not limited to, placenta extracts, beta glucan, fucoidan, sodium hyaluronate, and collagen.

Examples of anti-static agents includes, but is not limited to, methyl sulfonyl methane.

An example of bulking agent includes, but is not limited to, polypropylene A.

Examples of film former include, but are not limited to, copolymer of 1-vinyl-2-pyrrolidone and vinyl acetate, and Polyquaternium-6.

### Applications

The composition of the invention can be administered orally, topically, parenterally, sub-cutaneously, epicutaneously, intra-dermically, transdermically, intramusculary, enterally, intranasally, intra-respiratory, intra-vascular, ophthalmic preparation, intra-vaginal, endo-urethral, or by nasal inhalation. In a particular embodiment, the composition of the invention is administered sub-cutaneously, epicutaneously, intra-dermically, transdermically, or topically, preferably topically. The composition of the invention may be administered by microneedles, or patches.

The composition may in particular be applied to mucosa, corneum, epidermis, dermis, epithelium, endothelium, skin, skin appendages, connective tissues, or bone tissues, preferably skin, skin appendages or mucosa.

In an embodiment, the composition of the invention is selected from a sunscreen composition, a cosmetic composition, a dermatological composition, and a therapeutic composition. In a preferred embodiment, the composition is a sunscreen or a cosmetic composition.

### Topical Composition

In a particular embodiment, the composition of the invention is a topical composition. The topical composition comprises at least one compound of formula (I) (or of formula (I-1), (I-1′), (I-2), (I-2′), (I-3), (I-3′) or (I-4)) as defined herein, and at least one topically acceptable excipient.

A “topically acceptable excipient”, as used herein, denotes an excipient suitable for a topical application. Such an excipient can be judiciously chosen by the skilled artisan, for instance among the excipients described above.

Said topical composition may be a dermatological composition, therapeutic composition and/or a cosmetic composition.

The topical composition may in particular be in the form of a suspension, a cream, a spray, an aerosol, a butter, a stick, a gel, an ointment, a lotion, a solution, a solid, an emulsion, a micro-emulsion, an oil, a lyophilizate, a milk, a powder, a paste, a wax, a mousse, a patch, a film, a micelle, a liposome, or a foam. The composition can be prepared according to processes known to the skilled artisan.

Preferably, the topical composition is selected from a cream, a spray, a gel, an ointment, a lotion, an emulsion, a foam, a suspension and a milk.

The topical composition may be applied to mucosa, corneum, epidermis, dermis, epithelium, endothelium, skin or skin appendages (e.g. hair and nails), preferably mucosa, skin or skin appendages.

### Cosmetic Composition

In another particular embodiment, the composition of the invention is a cosmetic composition.

Said cosmetic composition comprises at least one compound of formula (I) (or formula (I-1), (I-1′), (I-2), (I-2′), (I-3), (I-3′) or (I-4)) according to the invention, and at least one cosmetically acceptable excipient.

A “cosmetically acceptable excipient”, as used herein, denotes an excipient suitable for a cosmetic application. Such an excipient can be judiciously chosen by the skilled artisan, for instance among the excipients described above.

The cosmetic composition can be administered orally, topically, parenterally, sub-cutaneously, epicutaneously, intra-dermically, transdermically, intramusculary, enterally, intranasally, intra-respiratory, or by nasal inhalation. In a preferred embodiment, the cosmetic composition is administered topically.

Preferably, the cosmetic composition is a topical composition or a dermatological composition, more preferably a topical composition.

The cosmetic composition may in particular be applied to mucosa, corneum, epidermis, dermis, epithelium, endothelium, skin or skin appendages (e.g. hair and nails), preferably mucosa, skin or skin appendages.

The cosmetic composition may in particular be in the form of a suspension, a cream, a spray, an aerosol, a butter, a stick, a gel, an ointment, a lotion, a solution, a solid, an emulsion, a micro-emulsion, an oil, a lyophilizate, a milk, a powder, a paste, a wax, a mousse, a patch, a film, a micelle, a liposome, or a foam. The composition can be prepared according to processes known to the skilled artisan.

The composition of the invention may be particularly well-suited for combatting and/or reducing the signs of cutaneous ageing, such as the formation of wrinkles and/or fine lines, skin sagging, loss of firmness, loss of radiance and/or evenness of the complexion, and/or for reinforcing the skin barrier.

The signs of cutaneous ageing may be related to intrinsic factors that are age-related, but also extrinsic factors, in particular UV-light exposure.

An object of the invention relates to a cosmetic use of the composition of the invention for combatting and/or reducing the signs of cutaneous ageing, such as the formation of wrinkles and/or fine lines, skin sagging, loss of firmness, loss of radiance and/or evenness of the complexion, and/or for reinforcing the skin barrier.

Another object of the invention is cosmetic process for combatting and/or reducing the signs of cutaneous ageing, such as the formation of wrinkles and/or fine lines, skin sagging, loss of skin firmness, loss of radiance and/or evenness of the complexion, and/or for reinforcing the skin barrier, comprising applying topically to the skin or its appendages, a composition of the invention.

### Sunscreen

In another particular embodiment, the composition of the invention is a sunscreen composition.

The sunscreen composition is advantageously applied topically (i.e. a topical composition). The sunscreen composition may in particular be in the form of a suspension, a cream, a spray, an aerosol, a butter, a stick, a gel, an ointment, a lotion, a solution, a solid, an emulsion, a micro-emulsion, an oil, a lyophilizate, a milk, a powder, a paste, a wax, a mousse, a patch, a film, a micelle, a liposome, or a foam. In a particular embodiment, the sunscreen composition is a formulation sunscreen type butter.

### Therapeutic Composition

In a particular embodiment, the composition of the invention is a therapeutic composition and, more particularly, said composition is a pharmaceutical composition or veterinary composition.

The therapeutic composition comprises at least one compound of formula (I) (or formula (I-1), (I-1′), (I-2), (I-2′), (I-3), (I-3′) or (I-4)) according to the invention and at least one pharmaceutically acceptable excipient.

The term “pharmaceutically acceptable”, as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration. A “pharmaceutically acceptable excipient”, as used herein, refers to all components of a pharmaceutical or therapeutic composition which facilitate the manufacture, the preservation and/or the delivery of the composition in vivo. Pharmaceutically acceptable excipients include, but are not limited to, diluents, preservatives, binders, lubricants, disintegrators, swelling agents, fillers, stabilizers, and combinations thereof.

The therapeutic composition can be administered orally, topically, parenterally, sub-cutaneously, epicutaneously, intra-dermically, transdermically, intramusculary, enterally, intranasally, intra-respiratory, or by nasal inhalation. In a preferred embodiment, the therapeutic composition is administered topically.

In a particular embodiment, the therapeutic composition is applied to a tissue chosen from the skin, skin appendages, mucosa, corneum, epidermis, dermis, epithelium, endothelium, connective tissues, bone tissues, and combinations thereof, preferably skin, skin appendages, mucosa, corneum, epidermis, dermis, epithelium, and endothelium, and more preferably skin, skin appendages, and mucosa. In another embodiment, the therapeutic composition is applied to a circulating medium, such as blood or plasma.

In a particular embodiment, the therapeutic composition is a dermatological composition.

Another object of the invention is a compound of formula (I) (or (I-1), (I-1′), (I-2), (I-2′), (I-3), (I-3′) or (I-4)) according to the invention or a composition of the invention (in particular, a therapeutic composition), for use in the treatment and/or prevention of a skin, a mucosa, an eye cornea, or skin appendage disease or condition.

Preferably, said skin, mucosa, eye cornea or skin appendage disease or condition is chosen from lipodystrophy, keloid scars, acne, psoriasis, atopic dermatitis, actinic keratosis, rosacea, melasma, melanoma, Merker cell carcinoma, basal cell carcinoma, squamous cell carcinoma, scar treatments, wound healing, alopecia, vitiligo, urticaria (hives), cold sores, impetigo, eczema, rashes dermatitis, ichthyosis, warts, blisters, pruritus, gangrene, bruises, pustules, bacterial skin infections like leprosy, carbuncles, cellulitis, impetigo, fungal infections like Athlete's foot (intertrigo) and sporotrichosis, fungal nail infections, viral infection like herpes, sunburns, lice, scabies, pressure ulcer disinfection, pressure ulcer healing, vaginitis, bladder cancer, endometriosis, uveitis, cornea diseases, cornea keratitis, corneal herpes, keratoconus, corneal dystrophies, pharyngitis, cutaneous and mucosal allergies. More preferably, said skin, mucosa, eye cornea or skin appendage disease or condition is chosen from lipodystrophy, keloid scars, acne, psoriasis, atopic dermatitis, actinic keratosis, rosacea, melasma, melanoma, Merker cell carcinoma, basal cell carcinoma, squamous cell carcinoma, scar treatments, wound healing, alopecia, vitiligo, urticaria (hives), cold sores, impetigo, eczema, rashes dermatitis, ichthyosis, warts, blisters, pruritus, gangrene, bruises, pustules, bacterial skin infections like leprosy, carbuncles, cellulitis, impetigo, fungal infections like Athlete's foot (intertrigo) and sporotrichosis, fungal nail infections, viral infection like herpes, sunburns, lice, scabies, pressure ulcer disinfection and pressure ulcer healing.

Another object of the invention is a method for delivering at least one compound of formula (I) (or (I-1), (I-1′), (I-2), (I-2′), (I-3), (I-3′) or (I-4)) to a tissue of a subject in need thereof, comprising administering an effective amount of a composition of the invention. The present invention also provides a method of delivering at least one compound of formula (I) (or of formula (I-1), (I-1′), (I-2), (I-2′), (I-3), (I-3′) or (I-4)) to a tissue of a subject, comprising: topically administering to a subject in need thereof a therapeutically effective amount of any presently described compositions useful in treating a disease, disorder, or condition of the tissue.

Particularly, said tissue is chosen from the skin, skin appendages, corneum, epidermis, dermis, epithelium, endothelium, connective tissues, bone tissues, and combinations thereof.

Preferably, said tissue is the skin, a skin appendage, or a mucosa.

Another object of the invention is a method for treating or preventing a skin, a mucosa, an eye cornea or skin appendage disease or condition, comprising administering to a subject in need thereof, a composition of the invention (in particular, a therapeutic composition), said composition comprising at least one compound of formula (I) (or of formula (I-1), (I-1′), (I-2), (I-2′), (I-3), (I-3′) or (I-4)) as defined herein.

A further object of the invention is a method of treating or preventing a skin, a mucosa, an eye cornea or a skin appendage disease, disorder, or condition in a subject, comprising: topically administering to a subject in need thereof a therapeutically effective amount of any presently described compositions useful in treating a skin, a mucosa, or an eye cornea disease, disorder, or condition. In a particular embodiment, the skin, mucosa, or eye cornea disease, disorder, or condition is selected from lipodystrophy, keloid scars, acne, psoriasis, atopic dermatitis, actinic keratosis, rosacea, melasma, melanoma, Merker cell carcinoma, basal cell carcinoma, squamous cell carcinoma, scar treatments, wound healing, alopecia, vitiligo, urticaria (hives), cold sores, impetigo, eczema, rashes dermatitis, ichthyosis, warts, blisters, pruritus, gangrene, bruises, pustules, bacterial skin infections like leprosy, carbuncles, cellulitis, impetigo, fungal infections like Athlete's foot (intertrigo) and sporotrichosis, fungal nail infections, viral infection like herpes, sunburns, lice, scabies, pressure ulcer disinfection and pressure ulcer healing, uveitis, cornea diseases, cornea keratitis, corneal herpes, keratoconus, corneal dystrophies, pharyngitis, cutaneous and mucosal allergies.

For the purpose of clarity, any element or feature of any method or composition or process described herein, can be combined with any other element or feature of any other method or composition or process described herein.

Another object of the invention is a use of a compound of formula (I) (or of formula (I-1), (I-1′), (I-2), (I-2′), (I-3), (I-3′) or (I-4)) of the invention for making a composition for treating and/or preventing a skin, mucosa, eye cornea, skin appendage disease or condition.

Another object of the invention is a compound of formula (I) (or of formula (I-1), (I-1′), (I-2), (I-2′), (I-3), (I-3′) or (I-4)) of the invention or a composition of the invention (in particular, a therapeutic composition), for use in the treatment and/or prevention of a disease or condition selected in the group consisting of lipodystrophy, keloid scars, acne, psoriasis, atopic dermatitis, actinic keratosis, rosacea, melasma, melanoma, Merker cell carcinoma, basal cell carcinoma, squamous cell carcinoma, scar treatments, wound healing, alopecia, vitiligo, urticaria (hives), cold sores, impetigo, eczema, rashes dermatitis, ichthyosis, warts, blisters, pruritus, gangrene, bruises, pustules, bacterial skin infections like leprosy, carbuncles, cellulitis, impetigo, fungal infections like Athlete's foot (intertrigo) and sporotrichosis, fungal nail infections, viral infection like herpes, sunburns, lice, scabies, pressure ulcer disinfection and pressure ulcer healing, vaginitis, cancer such as bladder cancer, endometriosis, uveitis, cornea diseases, cornea keratitis, corneal herpes, keratoconus, corneal dystrophies, pharyngitis, cutaneous and mucosal allergies.

### Kit

The present invention also relates to a kit comprising:
a composition according to the invention,
a washing composition, and
optionally an instruction guide.

In a preferred embodiment, the composition according to the invention in the kit is a topical composition. In a more preferred embodiment, the composition according to the invention in the kit is a cosmetic or a sunscreen composition, preferably a sunscreen composition.

A “washing composition” refers to a composition which enables the removal of part or all of a composition according to the invention, previously applied to a tissue such as the skin, skin appendages or a mucosa of a subject. More specifically, when the composition comprises adhesive compounds of formula (I), such as compounds of formula (I-3), (I-3′) or (I-4), the washing composition may enable the removal of such adhesive compounds.

In particular, the washing composition may comprise at least one “washing agent” and, optionally one or more excipients. The “washing agent” refers to a chemical or biological agent which is able to break the bond between adhesive compounds adhered to a tissue and said tissue. The washing agent may be a protein, a peptide (e.g. glutathione), an amino acid, an enzyme (e.g. cathepsin B), a thiol (e.g. 2-mercaptoethanol, N-acetyl cysteine), a dithiol (e.g. dithiothreitol), a pH-modifier, an acid, a base, a solvent, a saline solution (e.g. sodium chloride solution) or a combination thereof. Said washing agent can be judiciously chosen by the skilled artisan, depending on the nature of the bond between adhesive compounds adhered to a tissue and said tissue.

In a particular embodiment, said washing composition is a powder, a shampoo, a soap, a lotion, a solution, a solid, a scrubbing, a scraper, a mousse, a foam, a syndet, a gel, a shower gel, a spray, a mist, a wax, a strip, an enzyme composition, a detergent composition or a woven or non-woven fabric.

### Other Applications —Photoprotection and Photoinstability

Another object of the invention is a use of at least one compound as defined herein (preferably a compound of formula (I-3), (I-3′) or (I-4)) for reducing photodegradation and/or photoinstability of a pharmaceutical active ingredient or a cosmetic. Another object of the invention is a composition as defined herein or a composition for use as defined herein (preferably comprising a compound of formula (I-3), (I-3′) or (I-4)), said composition being a pharmaceutical or a veterinary composition, and further comprising a pharmaceutical or veterinary active ingredient.

Another object of the invention is a composition as defined herein or a composition for use as defined herein (preferably comprising a compound of formula (I-3), (I-3′) or (I-4)), said composition being a cosmetic composition, and further comprising a cosmetic active ingredient.

The term “photodegradation” refers to a partial or total degradation induced by the light, in particular UV-light.

The term “photoinstability” refers to an instability induced by the light, in particular UV-light.

A “pharmaceutical active ingredient” includes, without limitation, physically, physiologically or pharmacologically active substances. A pharmaceutical active ingredient is a substance that can be used for the treatment (e.g., therapeutic agent, vaccine antigen or antigenic material), prevention (e.g., prophylactic agent, vaccine), diagnosis (e.g., diagnostic agent), cure or mitigation of disease or illness. An active agent may also be a substance which affects the structure or function of the body, or pro-drugs, which become biologically active or more active after they have been placed in a predetermined physiological environment.

A “cosmetic” is a substance used in cosmetics uses, methods and processes, such as a sunscreen, a dye, a fragrance, a deodorant, a microbiote modulator, a skin modifier, and a skin lipid modulator.

In a particular embodiment, the at least one compound used for reducing photodegradation and/or photoinstability of a pharmaceutical active ingredient or a cosmetic, is in a form of a micelle or a liposome. Said pharmaceutical active ingredient or cosmetic may, in particular, be encapsulated within the at least one compound of the invention in a form of a micelle or a liposome.

A further object of the invention is a material comprising a support and at least one compound as defined herein. When the at least one compound as defined herein is adhesive, for instance a compound of formula (I-3), (I-3′) or (I-4), said compound is advantageously adhered to said support.

The support may be made of any organic and/or inorganic matter. In a particular embodiment, the support is a natural or synthetic polymeric support, a natural or synthetic fiber support, a stone, a metal, a plastic, a rubber, a glass support, or a paint (or equivalently “paint composition”).

The invention will also be described in further detail in the following examples, which are not intended to limit the scope of this invention, as defined by the attached claims.

### EXAMPLES

### Example 1. Preparation of Compounds of Formula (I), Physical Properties, and Skin Protection Studies

### Compound #1. 2-[(2-cyano-3,3-diphenyl-prop-2-enoyl)amino]ethyl 2-[4-(diethylamino)-2-hydroxy-benzoyl]benzoate

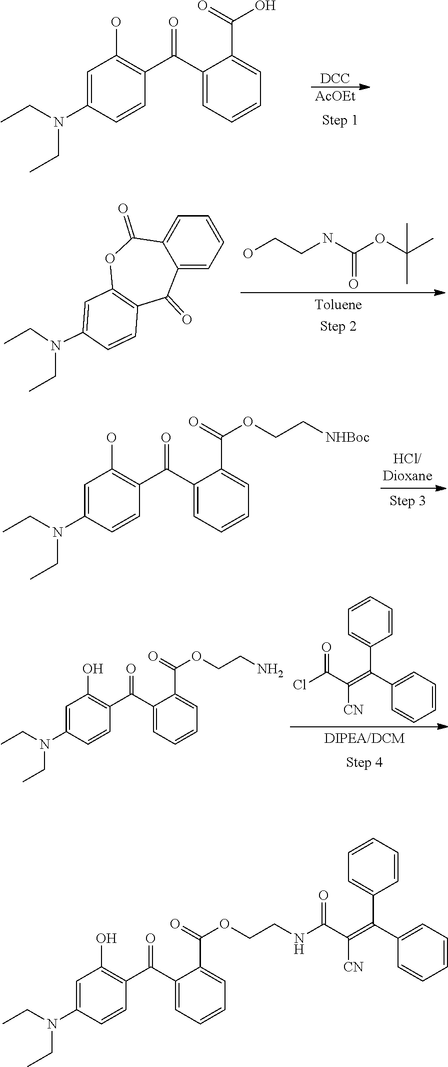

### Step 1:

In a 10 L double jacketed glass reactor under N₂ equipped with mechanical stirring, was charged 2-(4-(diethylamino)-2-hydroxybenzoyl)benzoic acid (500.0 g, 1.60 mol, 1.0 eq) followed by EtOAc (3.9 L). To this heterogeneous pink mixture was added a solution of DCC (362.2 g, 1.75 mol, 1.10 eq) in EtOAc (1.1 L) over a period of 10 minutes at 23° C. A temperature rise to 27° C. was noticed and the reaction mixture turned brown. The reaction mixture was stirred at 23° C. for 1.5 hours. An aliquot was taken after 1.5 hours, diluted with AcCN and analyzed by HPLC/MS. Incomplete conversion was observed. Extra DCC (10.0 g, 0.05 mol, 0.03 eq) solubilized in EtOAc was added and stirring was pursued for 18 hours. No significant improvement was observed. The reaction mixture was filtered through a 46 μm filter media and the filtrate was concentrated under reduced pressure to afford the desired oily residue (532.0 g, quant.) as a brown oil which was used as such for the next step.

¹H NMR 400 MHz (DMSO d₆) δ ppm 1.13 (t, 6H), 3.44 (m, 4H), 6.48 (d, 1H), 6.69 (dd, 1H), 7.65 (d, 1H), 7.81-7.87 (m, 2H), 7.92 (dd, 1H), 8.10 (dd, 1H)

### Step 2:

In a 10 L double jacketed glass reactor under N₂ equipped with mechanical stirring, was charged the previous compound (471.4 g, 1.60 mol, 1.0 eq) followed by toluene (4.0 L). A solution of N-Boc-ethanolamine (246.9 mL, 1.60 mol, 1.0 eq) in toluene (700.0 mL) was added at 23° C. The reaction mixture was heated to 110° C. and stirred at this temperature for 6 hours. Incomplete conversion was observed with 12% remaining starting material. Extra N-Boc-ethanolamine (24.7 mL, 0.16 mol, 0.10 eq) was added and stirring under reflux was pursued for 1 hour. The reaction mixture was then allowed to cool to 23° C. over 18 hours. Complete conversion was observed. The reaction mixture was hydrolyzed with water (2.0 L). Phases were separated and the organic layer was washed with 1M aqueous NaHCO₃ solution (2.5 L) and then with water (2.0 L). The organic layer was dried over MgSO₄, filtered, and evaporated to dryness under vacuum to afford (673.0 g, 92%) as an orange oil.

¹H NMR 400 MHz (DMSO d₆) δ ppm 1.11 (t, 6H), 1.37 (s, 9H), 3.16 (t, 2H), 3.39 (m, 4H), 4.07 (t, 2H), 6.09 (d, 1H), 6.19 (dd, 1H), 6.82 (d, 1H), 6.83 (tb, 1H), 7.43 (dd, 1H), 7.64 (d, 1H), 7.72 (m, 2H), 8.07 (dd, 1H), 12.51 (s, 1H)

### Step 3:

In a 20 L double jacketed glass reactor under N2 equipped with mechanical stirring, was charged the previous compound (583.0 g, 1.28 mol, 1.0 eq) followed by dioxane (4.18 L) and diethyl Ether (1.75 L). The solution was heated to 60° C. and a 4N HCl solution in dioxane (1.59 L, 6.38 mol, 5.0 eq) was added in 45 min. Precipitation started during the addition and some solid stuck to the reactor's walls. 1.0 L of dioxane was added and temperature was raised to 70° C. The suspension was stirred at 70° C. for 3 hours. The suspension was allowed to cool to 23° C. over 18 hours. The solid was collected by filtration washed with a 1:1 dioxane/diethyl ether mixture (1.6 L) and dried under vacuum at 45° C. for 18 hours to afford the desired compound (578.0 g, 98% yield) as a light pink solid.

¹H NMR 400 MHz (DMSO d₆) δ ppm 1.11 (t, 6H), 3.11 (t, 2H), 3.39 (q, 4H), 4.32 (t, 2H), 6.11 (d, 1H), 6.19 (dd, 1H), 6.80 (d, 1H), 6.83 (tb, 1H), 7.43 (dd, 1H), 7.64 (d, 1H), 7.67 (dd, 1H), 7.75 (t, 1H), 8.20 (d, 1H), 8.31 (sb, 2H).

### Step 4:

In a three-neck round-bottom flask, 2-cyano-3,3-diphenylprop-2-enoyl chloride (1.63 g, 6.0 mmol) prepared from 2-cyano-3,3-diphenylprop-2-enoic acid (1.5 g, 6.0 mmol), COCl₂ (1 eq.) in DCM and cat. DMF was added to a solution the previous amine (2.36 g, 6.0 mmol, 1.0 eq.) in DCM (20 mL) and DIPEA (1.1 mL). The solution was stirred at room temperature for 4 h and water (20 mL) was added. Phases were separated and the organic layer was washed with 1M aqueous NaHCO₃ solution (20 mL) and with water (20 mL). The organic layer was dried over MgSO4, filtered, and evaporated to dryness under vacuum. Precipitation with DIPE afforded (2.7 g, 76%) as an off-white solid.

¹H NMR 400 MHz (DMSO-d₆) δ ppm 1.13 (t, 6H), 3.25 (t, 2H), 3.39 (m, 4H), 3.60 (t, 2H), 6.09 (d, 1H), 6.19 (dd, 1H), 6.82 (d, 1H), 7.14-8.07 (m, 15H), 12.5 (s, 1H).

### Compound #2. 8-[[2-[4-(diethylamino)-2-hydroxy-benzoyl]benzoyl]amino]octyl 2-cyano-3,3-diphenyl-prop-2-enoate (OOD)

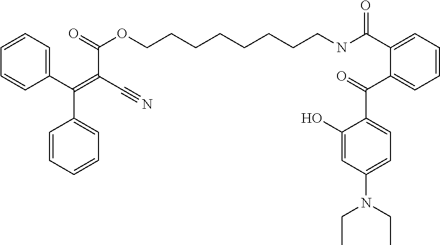 [SMILES: CCN(CC)c1ccc(C(=O)c2ccccc2C(=O)NCCCCCCCCOC(=O)C(C#N)=C(c2ccccc2)c2ccccc2)c(O)c1]

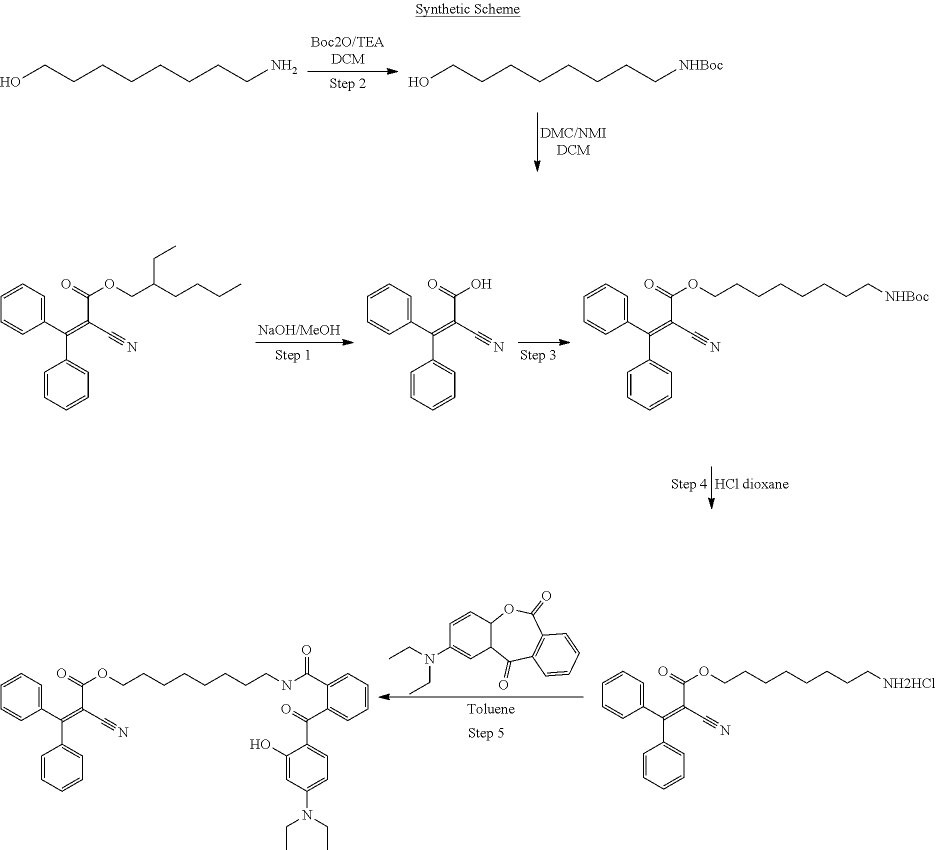

### Step 1:

In a 2-liter, round bottom three necked glass reactor, equipped with mechanical stirring and nitrogen flow, were added Octocrylene (190.3 mL, 0.553 mol, 1.0 eq) [BASF 51627391; 38128309T0], MeOH (1052.6 mL) and 30% aq. NaOH solution (49.2 mL, 1.660 mol, 3.0 eq). This solution was heated near 50° C. HPLC control was performed after 1 h30 and showed the reaction almost completed. The reaction was maintained for 1 h showing the same chromatogram profile. Then the solution was concentrated in a rotary evaporator till low volume, crystallization occurred during the night. The solid was stirred in 500 mL of a mixture of heptane/EtOAc: 90/10 during 1 hour, filtered, washed with heptane, then dried in an oven at 50° C. to afford a white powder 158 g as sodium salt.

The solid was diluted with nearly 1500 mL of water and HCl 37% was added until acid pH was reached. 500 mL of water were added and the suspension was stirred at room temperature for 1 h. The solid was filtered, washed with water until neutral pH, dried and finally washed with DIE. Drying was completed in an oven at 50° C. overnight under vacuum to afford 127.0 g, 0.509 mol, 92.1%) of the desired compound as a white powder.

### Step 2:

In a 500 mL round bottom flask was introduced 8-amino-octanol (10.5 g, 0.072 mol, 1.0 eq) followed by DCM (140.0 mL) and Et3N (11.1 mL, 0.080 mol, 1.1 eq). To this suspension was added dropwise at 23° C. a solution of Boc2O (16.566 g, 0.076 mol, 1.050 eq) in DCM (30 mL). LCMS analysis after 45 min showed a full conversion. The solution was diluted with DCM and washed with 0.5M aq HCl and then with water. The organic layer was dried over Na2SO4, filtered and concentrated under vacuum to afford 19.8 g of a clear oil containing tert-butanol. The product was left overnight and crystallization occurred. The heptane was added and the solid was triturated for 3 h, collected by filtration and dried at 40° C. under vacuum for 20 h to afford the desired product (17.6 g, 0.072 mol, 99.2%) as a white solid.

¹H NMR 400 MHz (DMSO-d6) d ppm 1.21-1.42 (m, 21H), 2.88 (q, 2H), 3.37 (q, 2H), 4.31 (t, 1H), 6.75 (t, 1H)

### Step 3:

In a 250 mL three neck round bottom flask was introduced the previous compound (10.0 g, 0.041 mol, 1.0 eq) followed by DCM (150.0 mL). To this solution was added the compound of step 1 (10.159 g, 0.041 mol, 1.0 eq), N-methylimidazole (9.7 mL, 0.122 mol, 3.0 eq) and 5 minutes later DMC (8.268 g, 0.049 mol, 1.2 eq). After 20 minutes HPLC control showed complete and clean conversion. The solution was diluted with DCM and washed with water. The organic layer was diluted with EtOAc and partially concentrated under vacuum to remove DCM. The residue was diluted with EtOAc. The solution was washed with 0.5M NaOH aq solution, 0.5M HCl aq solution and water successively, dried over Na2SO4, filtered and concentrated under vacuum to afford the desired compound (21.0 g, 0.040 mol, 98.9%) as a yellow oil.

¹H NMR 400 MHz (DMSO-d6) d ppm 1.02-1.21 (m, 10H), 1.32-1.37 (m, 11H), 2.89 (q, 2H), 4.02 (m, 2H), 6.75 (t, 1H), 7.18 (dd, 2H), 7.4-7.6 (m, 8H)

### Step 4:

In a 100 mL three neck round bottom flask was introduced the previous compound (10.4 g, 0.022 mol, 1.0 eq) followed by dioxane (104.0 mL). To this solution was added HCl 4M in dioxane (27.3 mL, 0.109 mol, 5.0 eq) and the reaction mixture stirred at 23° C. overnight. The solution was concentrated under vacuum affording the desired product as a yellowish oil. ¹H NMR 400 MHz (DMSO-d6) d ppm 1.07-1.53 (m, 12H), 2.75 (m, 2H), 4.02 (t, 2H), 7.18 (dd, 2H), 7.39-7.57 (m, 8H), 7.87 (bs, 2H)

### Step 5:

In a 250 mL three neck round bottom flask was introduced the previous compound (6.0 g, 0.012 mol, 1.0 eq) followed by toluene (60.0 mL). 2-(diethylamino)-4a, 11a-dihydrobenzo[c][1]benzoxepine-6,11-dione (3.731 g, 0.012 mol, 1.0 eq) was solubilized in 40 mL of toluene and added to the previous solution at 23° C. The reaction mixture was heated to 50° C. At about 40° C., DIPEA (3.0 mL, 0.017 mol, 1.5 eq) was added and the solution was stirred at this temperature for 4 hours. After cooling at room temperature, the solution was diluted with EtOAc, washed with a sat. NH₄Cl solution and with water. The organic layer was dried over Na2SO4, filtered, and concentrated under vacuum. Purification by flash chromatography using 30/70 EtOAc/Heptane then 50/50 EtOAc/Heptane afforded 7 g as an amorphous yellowish solid containing 1.7% of DCU. The residue was taken up into DCM and a slight precipitate (DCU) was filtrated. A second silica column purification was performed using DCM 100%, 30:70 EtOAc/Heptane then 50:50 EtOAc/Heptane. The product was concentrated under vacuum and taken up into acetone, filtered to remove solid DCU and concentrated under vacuum to afford the desired compound (6.0 g, 0.009 mol, 76.8%) as a yellowish amorphous solid.

¹H NMR 400 MHz (DMSO-d6) d ppm 1.16 (m, 14H), 1.43 (m, 4H), 3.2 (q, 2H), 3.42 (q, 4H), 4.06 (t, 2H), 6.12 (d, 1H), 6.17 (d, 1H), 6.75 (t, 1H), 6.96 (d, 1H), 7.19 (d, 2H), 7.38-7.67 (m, 12H), 12.6 (s, 1H)

### UV Absorbance Profil of Compound #2 (OOD)

Compound #2 (OOD), Univul A plus (DHHB)+Octocrylene (OCR) were dissolved in a solvent at the same concentration (125 μM). Absorbance spectra was read with FLUOstar plate reader and results are shown in the following table 1.

TABLE 1

| Solvent | OOD λmax | DHHB + OCR λmax |
|---|---|---|
| Ethanol | 356 | 358 |
| DMSO | 356 | 355 |
| Cetiol B | 355 | 353 |
| Cetiol HE | 355 | 351 |
| Dubdis | 356 | 351 |

λmax absorbance value of the multifilter OOD (Compound #2) is more stable when solubilized in five different solvents (Ethanol (λmax:356 nm), DMSO (λmax:356 nm), Cetiol B (Dibutyl Adipate) (λmax:355 nm), Cetiol HE (PEG-7 Glyceryl Cocoate) (λmax:355 nm), DubDis (diisopropyl sebacate) (λmax:355 nm) than the mix of DHHB+OCR (Ethanol (λmax:358 nm), DMSO (λmax:355 nm), Cetiol B (λmax:353 nm), Cetiol HE (λmax:351 nm), DubDis (diisopropyl sebacate) (λmax:351 nm) at the same molar concentration.

Photostability of Compound #2 (OOD) after UV irradiation

Compound #2 (OOD), Univul A plus (DHHB), Octocrylene (OCR), and Univul A plus (DHHB)+Octocrylene (OCR) were dissolved in Ethanol at 1 mM and were irradiated at 2000 and 4000 J/dm2 for 1 h and 2 h in a 24-well plate, respectively, in 2 replicates (1 replicate for HPLC and 1 replicate for absorbance spectra). Absorbance spectra was read with FLUOstar plate reader and results are shown in the following table 2.

TABLE 2

| | Without UV | Irradiation 2000 | Irradiation 4000 |
|---|---|---|---|
| Absorbance peak | irradiation | J/dm2 | J/dm2 |
| OCR | 304 nm | 304 nm | 304 nm |
| DHHB | 357 nm | 357 nm | 357 nm |
| OCR + DHHB | 354 nm | 356 nm | 356 nm |
| Compound #2 | 357 nm | 357 nm | 357 nm |
| (ODD) | | | |

An irradiation on the UVB and UVA range at 2000 or 4000 J/dm2 for 1 or 2 hours in ethanol did not modify the UV absorbance profiles of OOD and the mix DHHB+OCR. The λmax of OOD is 357 nm before and after irradiation and the λmax of the mix DHHB+OCR goes from 354 nm before irradiation to 356 nm after irradiation, which is negligible.

No degradation of OOD in Ethanol was observed by HPLC after irradiation (purity: 98.80%) compared to non-irradiated OOD (purity: 99.17%). No degradation of DHHB, OCR nor mixed DHHB+OCR in Ethanol was observed by HPLC after irradiation.

Solubility of Compound #2 (OOD) in sunscreen solvents:

### Material and Methods

Compound #2 (OOD) and a sunscreen solvent were weighed in a Becher. The Becher was placed on a magnetic stir plate, heated up to 50° C., and the solution was vigorously mixed with a bar magnet.

OOD is soluble in Dubdis (Diisopropyl sebacate, Stearinerie Dubois) at 50° C. at a 1/1 w/w, in Cetiol B (Dibutyl Adipate, BASF) at 50° C. at a 1/1 w/w and in Cetiol HE (PEG-7 Glyceryl Cocoate, BASF) at 50° C. at a ⅕ w/w.

UV Protection Performance of Compound #2 (OOD) UVB and UVA protections have been assessed ex vivo on pig skin by measuring the autofluorescence quenching. OOD (Compound #2), the mix of DHHB+OCR were solubilized at the same molar concentration in Dottisol (isosorbide dimethyl ether), covered 6.25 cm2 skin square and incubated 20 min or 2 hours at room temperatures, and then 7 mm skin punches were cut and placed in a black 96-well plate. UVB and UVA protections were measured after respectively an excitation at 300 nm and 355 nm and a fluorescence emission was read at 460 nm. DMSO was added on the pig skin before the reading of the residual fluorescence. The UV protection was measured after 2 hours of incubation at room temperature and results are shown in the following table 3

TABLE 3
Autofluorescence of pig skin after 2 h of incubation with
OOD - UVA/UVB (Mean +/− SD, N = 3)

| | UVA | UVB |
|---|---|---|
| | Fluorescence | Fluorescence |
| | (Ex355/Em460) | (Ex300/Em460) |
| OCR + DHHB | 2391 +/− 99 | 11394 +/− 591 |
| Compound #2 (ODD) | 2881 +/− 617 | 15248 +/− 3227 |
| Blank | 192448 +/− 27816 | 208969 +/− 22082 |

The results show that UVA and UVB protections of the pig skin provided by compound #2 (OOD) are similar compared to the mix OCR+DHHB.

### Compound #3. 2-[[4-[2-(2,5-dioxopyrrol-1-yl)ethoxy]-6-[2-[(2-hydroxybenzoyl)amino]ethylamino]-1,3,5-triazin-2-yl]amino]ethyl 2-[4-(diethylamino)-2-hydroxy-benzoyl]benzoate

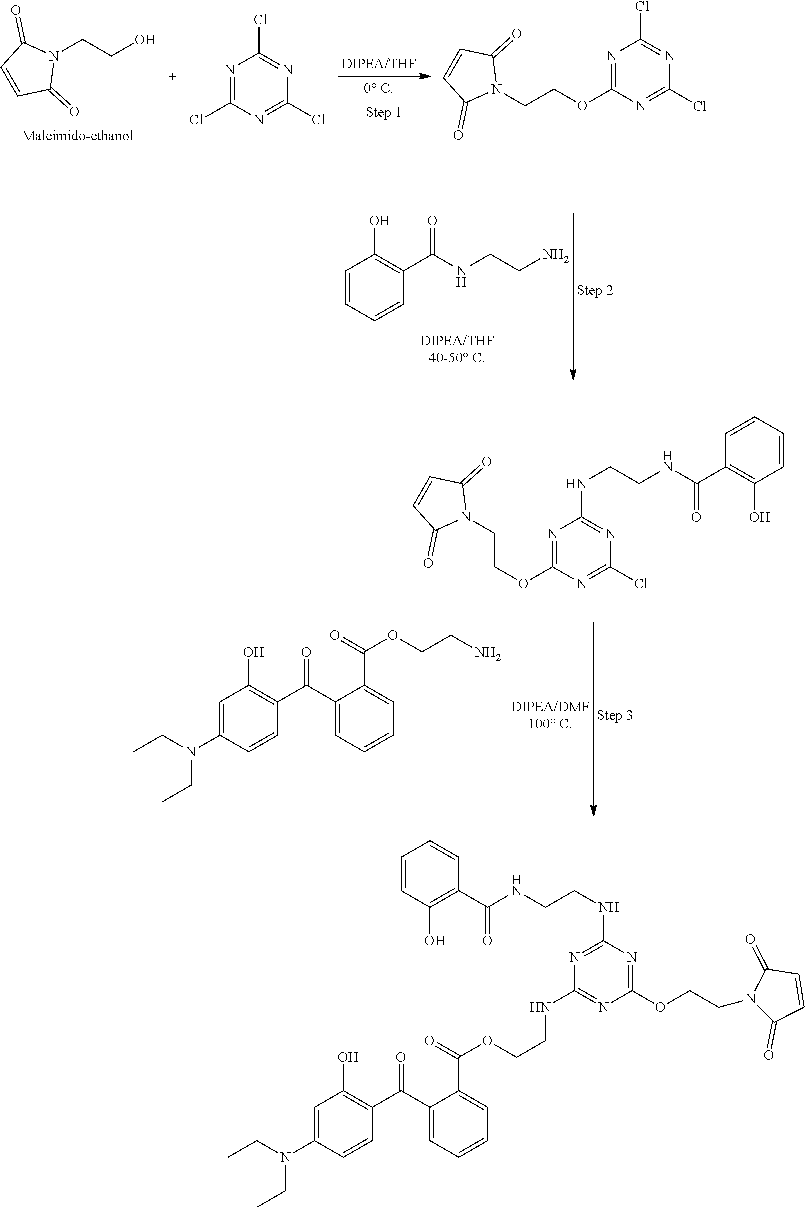

### Step 1:

The product 2-N-maleimidoethoxy-4,6-dichloro-1,3,5-triazine of step 1 was prepared as described in “Alternating Radical Ring-Opening Polymerization of Cyclic Ketene Acetals: Access to Tunable and Functional Polyester Copolymers” Megan R. Hill, Tomohiro Kubo, Sofia L. Goodrich, C. Adrian Figg, and Brent S. Sumerlin, Macromolecules 2018, 51, 14, 5079-5084.

### Step 2:

N-(2-Aminoethyl)-2-hydroxybenzamide (CAS 36288-93-4) was purchased from Combi-Blocks.

To a solution of previous compound (1.45 g, 5 mmol) in dry THE (20 mL) were added DIPEA (1 mL, 5.8 mmol) and N-(2-Aminoethyl)-2-hydroxybenzamide (0.92 g, 5.1 mmol). The reaction mixture was stirred at 50° C. for 2d. The mixture was filtered through a pad of silica gel, and the solvent was removed under reduced pressure. The crude product was purified by flash chromatography (silica gel, hexane/EtOAc 6:4) to give 0.72 g (33%) of the desired compound as an off-white solid.

¹H NMR 400 MHz (DMSO d₆) δ ppm 3.41 (t, 4H), 4.02 (t, 2H), 4.68 (t, 2H), 7.0 (s, 2H), 7.02 (d, 1H), 7.28 (dd, 1H), 7.45 (d, 1H), 7.87 (dd, 1H).

### Step 3:

A solution of the previous compound (0.7 g, 1.6 mmol) and 2-[4-(diethylamino)-2-hydroxybenzoyl]benzoate 2-aminoethyl ester (0.57 g, 1.6 mmol) and DIPEA (0.3 mL) in DMF (15 mL) were heated at 100° C. overnight. The mixture was cooled, concentrated under vacuum, and partitioned between CH₂Cl₂ (50 mL) and H₂O (50 mL). The layers were separated and the organic phase was dried (Na₂SO₄), filtered, and concentrated. The residue was purified by flash chromatography (silica gel, CH₂Cl₂/MeOH from 100:0 to 90:10 to give 0.49 g (41%) of the desired compound as an off-white solid.

¹H NMR 400 MHz (DMSO d₆) δ ppm 1.13 (t, 6H), 3.25 (dd, 2H), 3.39-3.41 (m, 8H), 3.60 (t, 2H), 4.04 (t, 2H), 4.6-4.7 (m, 4H), 6.09 (d, 1H), 6.19 (dd, 1H), 6.82 (d, 1H), 7.0 (s, 2H), 7.1-7.3 (m, 2H), 7.44 (d, 1H), 7.6-7.7 (m, 4H), 7.8 (dd, 2H), 12.5 (s, 1H).

### Compound #4. 6-[[2-[4-(diethylamino)-2-hydroxy-benzoyl]benzoyl]amino]hexyl 2-cyano-3,3-diphenyl-prop-2-enoate (OHD)

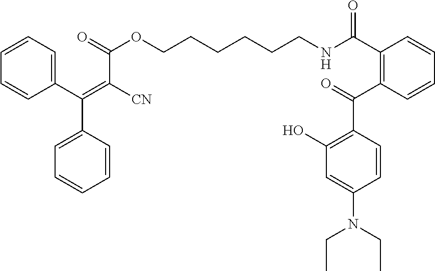 [SMILES: CCN(CC)c1ccc(C(=O)c2ccccc2C(=O)NCCCCCCOC(=O)C(C#N)=C(c2ccccc2)c2ccccc2)c(O)c1]

Compound #4 was prepared as described in the synthetic scheme of compound #2 using Boc-NH-hexanol and was obtained as yellow solid with 58% yield.

¹H NMR 400 MHz (CD₃CN) δ ppm 12.64 (s, 1H), 7.63 (m, 1H), 7.53 (m, 3H), 7.45 (m, 3H), 7.38 (m, 5H), 7.15 (ddd, J=6.9, 3.4, 1.8 Hz, 2H), 6.95 (t, 1H), 6.93 (d, J=9.1 Hz, 1H), 6.13 (dd, J=9.2, 2.6 Hz, 1H), 6.08 (d, J=2.5 Hz, 1H), 4.01 (t, J=6.4 Hz, 2H), 3.38 (q, J=7.1 Hz, 4H), 3.13 (dd, J=13.0, 6.9 Hz, 2H), 1.42-1.24 (m, 5H), 1.18-0.97 (m, 11H).

### UV Absorbance Profil of Compound #4 (OHD)

Compound #4 (OHD), Univul A plus (DHHB)+Octocrylene (OCR) were dissolved in a solvent at the same concentration (125 μM). Absorbance spectra was read with FLUOstar plate reader and results are shown in the following table 4.

TABLE 4

| Solvent | OHD λmax | DHHB + OCR λmax |
|---|---|---|
| Ethanol | 356 | 358 |
| DMSO | 356 | 355 |

λmax absorbance value of the multifilter OHD (Compound #4) is more stable when solubilized in two different solvents (Ethanol (λmax:356 nm) and DMSO (λmax:356 nm) than the mix of DHHB+OCR (Ethanol (λmax:358 nm) and DMSO (λmax:355 nm at the same molar concentration.

Photostability of Compound #4 (OHD) after UV Irradiation

Compound #4 (OHD), Univul A plus (DHHB), Octocrylene (OCR), and Univul A plus (DHHB)+Octocrylene (OCR) were dissolved in Ethanol at 1 mM and were irradiated at 2000 and 4000 J/dm2 for 1 h and 2 h in a 24-well plate, respectively, in 2 replicates (1 replicate for HPLC and 1 replicate for absorbance spectra). Absorbance spectra was read with FLUOstar plate reader and results are shown in the following table 5.

TABLE 5

| | Without UV | Irradiation 2000 | Irradiation 4000 |
|---|---|---|---|
| Absorbance peak | irradiation | J/dm2 | J/dm2 |
| OCR | 304 nm | 304 nm | 304 nm |
| DHHB | 357 nm | 357 nm | 357 nm |
| OCR + DHHB | 354 nm | 356 nm | 356 nm |
| Compound #4 | 358 nm | 357 nm | 357 nm |
| (OHD) | | | |

An irradiation on the UVB and UVA range at 2000 or 4000 J/dm2 for 1 or 2 hours in ethanol did not modify the UV absorbance profiles of OHD and the mix DHHB+OCR. The λmax of OHD is 358 nm before irradiation and 357 nm after irradiation and the λmax of the mix DHHB+OCR goes from 354 nm before irradiation to 356 nm after irradiation, which is negligible. No degradation of OHD in Ethanol was observed by HPLC after irradiation (purity: 97.84%) compared to non-irradiated OHD (purity: 98.13%). No degradation of DHHB, OCR nor mixed DHHB+OCR in Ethanol was observed by HPLC after irradiation.

### Solubility of Compound #4 (OHD) in Sunscreen Solvents:

### Material and Methods

Compound #4 (OHD) and a sunscreen solvent were weighed in a Becher. The Becher was placed on a magnetic stir plate, heated up to 50° C., and the solution was vigorously mixed with a bar magnet.

OHD is soluble in Dottisol (dimethyl isosorbide) at 50° C. at a 1/1.7 w/w. OHD is also soluble in Dimethyl capramide (Spectrasolv DMDA, Hallstar) at 50° C. at a 1/1 w/w, and in Cetiol B (Dibutyl Adipate, BASF) at 50° C. at a ⅓ w/w.

### Compound #5. 2-cyano-N-(8-(5-((2-ethylhexyl)oxy)-2-(4-(4-((2-ethylhexyl)oxy)-2-hydroxyphenyl)-6-(4-methoxyphenyl)-1,3,5-triazin-2-yl)phenoxy)octyl)-3,3-diphenylacrylamide (BOO)

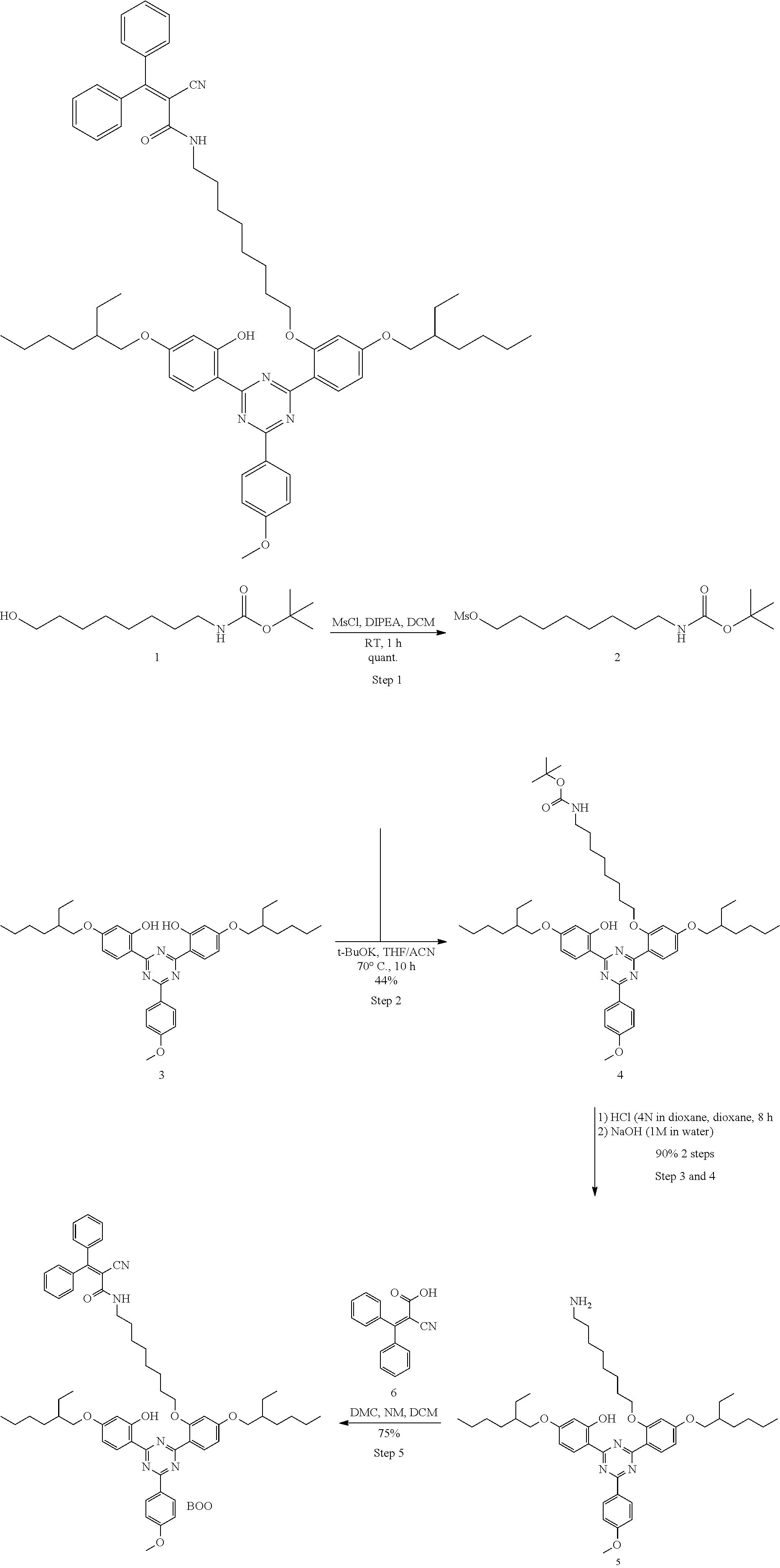

### Step 1:

In a 250 mL three necked round bottom flask, was introduced N-Boc-aminooctanol (10.0 g, 0.041 mol, 1.0 eq) followed by DCM (100.0 mL). At 23° C., DIPEA (7.5 mL, 0.043 mol, 1.05 eq) was added to this solution. 5 min later was added a solution of methanesulfonyl chloride (3.2 mL, 0.041 mol, 1.0 eq) previously diluted in DCM (12.5 mL) keeping temperature below 15° C. The reaction solution was stirred at 23° C. for 45 min. Reaction solution was hydrolyzed with 50 mL of 0.2M HCl aqueous solution before dilution with water and DCM. Liquid/liquid extraction was performed, and the recovered organic layer was washed with water twice, dried over Na2SO4, filtered and concentrated under vacuum affording the desired product (13.8 g, 0.041 mol, quantitative yield) as a yellowish oil which slowly crystallized at 23° C.

### Step 2:

In a 250 mL three necked round bottom flask, was introduced Tinosorb-S (5.00 g, 0.008 mol, 1.0 eq) followed by MeCN (88.2 mL) and THE (58.8 mL). To this solution was added 8-((tert-butoxycarbonyl)amino)octyl methanesulfonate (4.39 g, 0.012 mol, 1.5 eq) and tBuOK (1.61 g, 0.014 mol, 1.8 eq) leading to an orange solution. This mixture was then heated up to 70° C. for 3 hours, TLC revealed uncomplete conversion. Mixture was stirred at 23° C. for 18 h and then heated back to 70° C. for 9 extra hours. The reaction mixture was hydrolyzed with 70 mL of a saturated NH₄Cl solution and diluted with EtOAc and water. Liquid/liquid extraction was performed, and recovered organic layer was washed once with water and brine. Organic layer was dried over Na2SO4, filtered and concentrated under vacuum affording 7.3 g of an orange oil. Residue was taken up into a minimum of THE and purified by silica column chromatography, using Heptane/EtOAc (90:10 to 85:15) as eluent. Purest fractions were combined and concentrated under vacuum affording the desired product (3.0 g, 0.004 mol, 44% yield) as a yellowish oil.

### Steps 3 and 4:

In a 250 mL round bottom flask, was introduced the previous compound (3.00 g, 0.004 mol, 1.0 eq) followed by Dioxane (22.0 mL). To this solution was added 4M HCl in Dioxane (3.5 mL, 0.014 mol, 4.0 eq) and the solution was stirred at 80° C. for 6 h. The reaction mixture was allowed to come back at 23° C. The suspension was diluted with DIE leading to a complete precipitation. Suspension was filtered under N2 and the solid was washed once with DIE. The solid was dried under vacuum at 45° C. affording (2.50 g, 0.003 mol, 90% yield) as a yellowish solid. The solid was introduced in a 100 mL round bottom flask followed by 2-MeTHF(50.0 mL). To this yellow suspension was added 1M NaOH aqueous solution (15.8 mL, 0.016 mol, 5.0 eq). After complete solubilization, liquid/liquid extraction was performed, and organic layer recovered, washed with water and brine, dried over Na2SO4, filtered and concentrated under vacuum affording the desired product (2.40 g, 0.003 mol, quantitative yield) as a yellow oil.

### Step 5:

In a 100 mL three neck round bottom flask, was introduced the previous product (2.23 g, 0.003 mol, 1.0 eq) followed by DCM (23.0 mL). To this solution was added 2-cyano-3,3-diphenylacrylic acid (described page 58) (0.74 g, 0.003 mol, 1.0 eq) and 1-Methylimidazole (0.71 mL, 0.009 mol, 3.0 eq). After 5 minutes, DMC (0.599 g, 0.004 mol, 1.2 eq) was added and solution was stirred at 23° C. for 2 h. The reaction mixture was diluted with EtOAc and water. Liquid/liquid extraction was performed, and the organic layer was washed once with 1M NaHCO₃ aqueous solution, once with 0.5M HCl aqueous solution and once with water and brine. The organic layer was dried over Na2SO4, filtered and concentrated under vacuum affording 2.7 g as a yellowish oil. The oily residue was taken up into DCM and purified over a short silica pad eluting with EtOAc/Heptane (30:70 to 60:40). Purest fractions were concentrated under vacuum affording the desired product (2.19 g, 0.002 mol, 75% yield) as a yellowish amorphous solid.

1H NMR (400 MHz, DMSO) δ 13.7 (s, 1H), 8.6 (d, J=8.9 Hz, 2H), 8.5 (d, J=8.9 Hz, 1H), 8.4 (d, J=8.7 Hz, 2H), 7.5-7.5 (m, 3H), 7.4-7.3 (m, 5H), 7.2 (d, J=9.0 Hz, 2H), 7.1 (dd, J=7.4, 2.2 Hz, 2H), 6.8 (s, 1H), 6.8 (d, 1H), 6.6 (dd, J=9.0, 2.4 Hz, 1H), 6.5 (d, J=2.5 Hz, 1H), 4.2 (t, J=6.5 Hz, 2H), 4.0 (d, J=5.8 Hz, 2H), 3.9 (d, J=5.8 Hz, 2H), 3.9 (s, 3H), 2.9 (q, J=6.5 Hz, 2H), 1.8 (q, J=7.4 Hz, 2H), 1.8-1.6 (m, 2H), 1.5-1.2 (m, 18H), 1.1 (q, J=6.7 Hz, 2H), 1.1-0.9 (m, 4H), 0.9-0.8 (m, 12H), 0.8 (q, J=7.6 Hz, 2H).

UV absorbance profile of Compound #5 (BOO)

Compound #5 (BOO) and BEMT (6,6′-(6-(4-methoxyphenyl)-1,3,5-triazine-2,4-diyl)bis(3-((2-ethylhexyl)oxy)phenol)=Tinosorb S)+Octocrylene (OCR) were dissolved in a solvent at the same concentration (50 μM). Absorbance spectra was read with FLUOstar plate reader and results are shown in the following table 6.

TABLE 6

| Solvent | BOO λmax double peak | | BEMT + OCR λmax double peak | |
|---|---|---|---|---|
| Dottisol | 301 | 324 | 312 | 330 |
| Dubdis | 301 | 323 | 310 | 351 |

The λmax absorbance of the mix of BEMT-OCR shows a double peak at 310 and 351 nm when the λmax absorbance of the BOO shifts by 10 nm in UVB showing a double peak at 301 and 323 nm at the same molar concentration diluted in Dubdis (diisopropyl sebacate). The same shift in UVB is obtained in Dottisol, respectively 301 and 324 nm for BOO and 312 and 330 nm for the mix of BEMT-OCR at the same molar concentration.

Photostability of Compound #5 (BOO) after UV Irradiation

Compound #5 (BOO), Tinosorb S (BEMT), and Octocrylene (OCR) were dissolved in Dubdis at 2 mM and were irradiated at 4000 J/dm2 for 2 h in a 24-well plate, respectively, in 2 replicates (1 replicate for HPLC and 1 replicate for absorbance spectra). Absorbance spectra was read with FLUOstar plate reader and results are shown in the following table 7.

TABLE 7

| Absorbance peak | Without UV irradiation | Irradiation 4000 J/dm2 |
|---|---|---|
| OCR | 305 nm | 309 nm |
| BEMT | 310 and 351 nm | 310 and 351 nm |
| OCR + BEMT | 310 and 351 nm | 312 and 351 nm |
| Compound #5 (BOO) | 301 and 323 nm | 301 and 324 nm |

An irradiation on the UVB and UVA range at 4000 J/dm2 for 2 hours in Dubdis did not modify the UV absorbance profiles and λmax of BOO and the mix BEMT+OCR.

The λmax of BOO is 301 nm and 323 nm before and after irradiation and the λmax of the mix BEMT+OCR goes from 310 and 351 nm before irradiation to 312 and 351 nm after irradiation, which is negligible.

No degradation of BOO in Dubdis was observed by HPLC after irradiation (purity: 98.13%) compared to non-irradiated BOO (purity: 98.88%). No degradation of BEMT, OCR nor mixed BEMT+OCR in Dubdis was observed by HPLC after irradiation.

### Solubility of Compound #5 (BOO) in Sunscreen Solvents:

### Material and Methods

Compound #5 (BOO) and a sunscreen solvent were weighed in a Becher. The Becher was placed on a magnetic stir plate, heated up to 50° C., and the solution was vigorously mixed with a bar magnet.

BOO is soluble in Dubdis (Diisopropyl sebacate, Stearinerie Dubois) and Dottisol (dimethyl isosorbide) at 50° C. at a 1/1.5 w/w. BOO is also soluble in Dimethyl capramide (Spectrasolv DMDA, Hallstar) at 50° C. at a 1/1,6 w/w.

### UV Protection Performance of Compound #5 (BOO)

UVB and UVA protections have been assessed ex vivo on pig skin by measuring the autofluorescence quenching. BOO (Compound #5) and the mix of BEMT+OCR were solubilized at the same molar concentration in Dottisol (isosorbide dimethyl ether), covered 6.25 cm² skin square and incubated 4 hours at 34° C., washed or not with 10 mL Ethanol for 5 min, and then 7 mm diameter skin punches were cut and placed in a black 96-well plate. UVB and UVA protections were measured after respectively an excitation at 300 nm and 355 nm and a fluorescence emission was read at 460 nm. The UV protection was measured after 4 hours of incubation at room temperature and results are shown in the following table 8

TABLE 8
Autofluorescence of pig skin after 4 h of incubation with
BOO - UVA/UVB (Mean +/− SD, N = 3)

| | UVA | | UVB | |
|---|---|---|---|---|
| | Fluorescence (Ex355/Em460) | | Fluorescence (Ex300/Em460) | |
| | No Wash | EtOH Wash | No Wash | EtOH Wash |
| OCR + BEMT | 45880 +/− 8344 | 181062 +/− 16934 | 48393 +/− 7513 | 173793 +/− 15263 |
| Compound #5 | 49672 +/− 9882 | 217235 +/− 4204 | 49009 +/− 9100 | 205205 +/− 3116 |
| (BOO) | | | | |
| Blank | 211539 +/− 12853 | 214515 +/− 24373 | 205720 +/− 7700 | 201781 +/− 25373 |

The results show that UVA and UVB protections of the pig skin provided by compound #5 (BOO) are similar compared to the mix OCR+BEMT.

### Compound #6. 2-cyano-N-(8-(2-(4-(2-((8-(2,5-dioxo-2,5-dihydro-1H-pyrrol-1-yl)octyl)oxy)-4-((2-ethylhexyl)oxy)phenyl)-6-(4-methoxyphenyl)-1,3,5-triazin-2-yl)-5-((2-ethylhexyl)oxy)phenoxy)octyl)-3,3-diphenylacrylamide (BOO-OM)

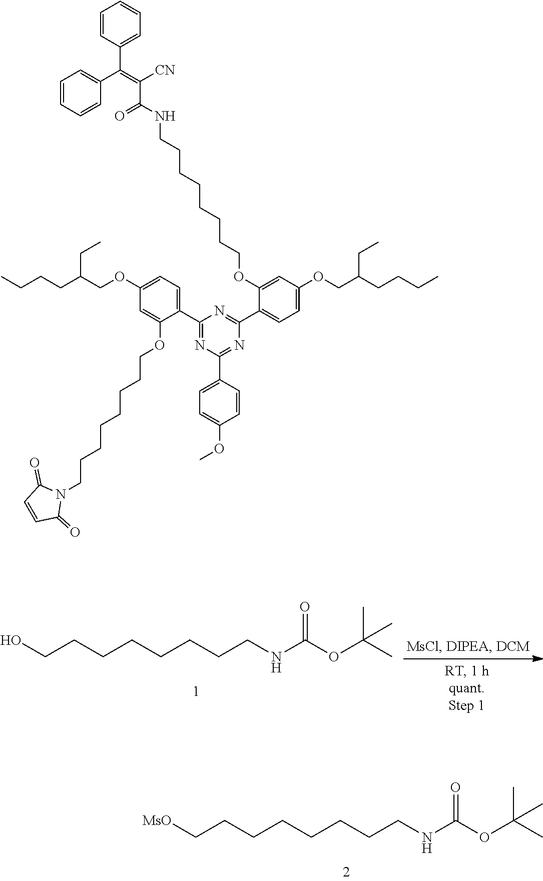 [SMILES: CCCCC(CC)COc1ccc(-c2nc(-c3ccc(OC)cc3)nc(-c3ccc(OCC(CC)CCCC)cc3OCCCCCCCCN3C(=O)C=CC3=O)n2)c(OCCCCCCCCNC(=O)C(C#N)=C(c2ccccc2)c2ccccc2)c1 ; CC(C)(C)OC(=O)NCCCCCCCCO ; CC(C)(C)OC(=O)NCCCCCCCCOS(C)(=O)=O]

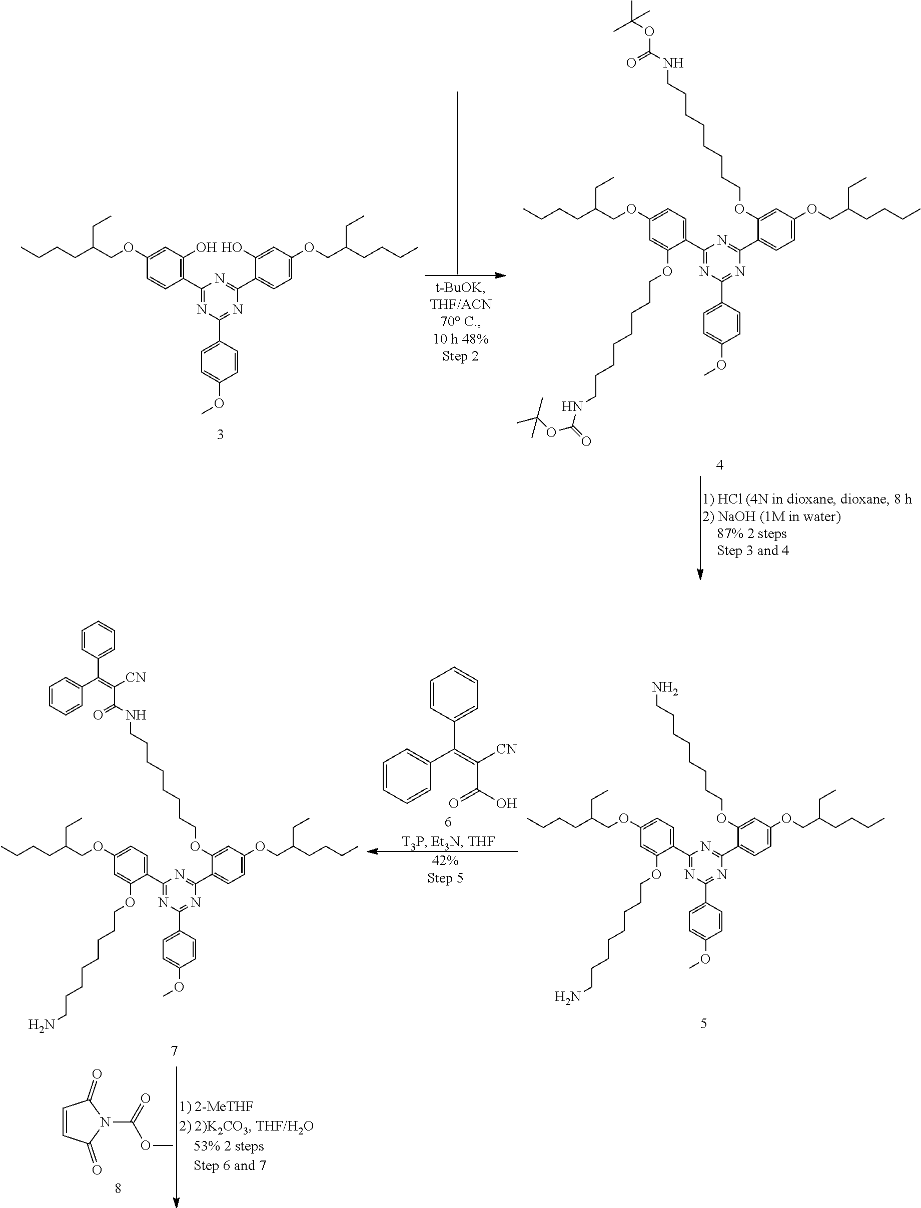 [SMILES: CCCCC(CC)COc1ccc(-c2nc(-c3ccc(OC)cc3)nc(-c3ccc(OCC(CC)CCCC)cc3O)n2)c(O)c1 ; CCCCC(CC)COc1ccc(-c2nc(-c3ccc(OC)cc3)nc(-c3ccc(OCC(CC)CCCC)cc3OCCCCCCCCNC(=O)OC(C)(C)C)n2)c(OCCCCCCCCNC(=O)OC(C)(C)C)c1 ; CCCCC(CC)COc1ccc(-c2nc(-c3ccc(OC)cc3)nc(-c3ccc(OCC(CC)CCCC)cc3OCCCCCCCCNC(=O)C(C#N)=C(c3ccccc3)c3ccccc3)n2)c(OCCCCCCCCN)c1 ; N#CC(C(=O)O)=C(c1ccccc1)c1ccccc1 ; CCCCC(CC)COc1ccc(-c2nc(-c3ccc(OC)cc3)nc(-c3ccc(OCC(CC)CCCC)cc3OCCCCCCCCN)n2)c(OCCCCCCCCN)c1 ; COC(=O)N1C(=O)C=CC1=O]

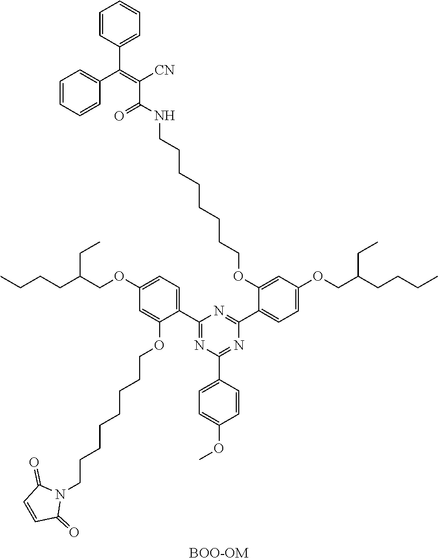

### Step 1:

In a 250 mL round bottom three necked flask, was introduced N-Boc-aminooctanol (15.0 g, 0.061 mol, 1.0 eq) followed by DCM (150.0 mL). At 23° C. was added DIPEA (11.2 mL, 0.064 mol, 1.05 eq). Finally, a solution of methanesulfonyl chloride (4.7 mL, 0.061 mol, 1.0 eq) previously diluted in DCM (18.8 mL) was added keeping the temperature below 15° C. The solution was then stirred at room temperature for 3 h. The reaction mixture was hydrolyzed with a 0.5M HCl aqueous solution. Mixture was diluted with DCM and water before liquid/liquid extraction. The recovered organic layer was washed with water, dried over Na2SO4, filtered and concentrated under vacuum affording the desired product (20.0 g, 0.062 mol, quantitative yield) as a slightly yellowish oil which slowly crystallized at 23° C.

### Step 2:

In a 500 mL three neck round bottom flask, was introduced Tinosorb-S(9.00 g, 0.014 mol, 1.0 eq) followed with MeCN (158.8 mL) and THE (105.9 mL). To this solution was added 8-((tert-butoxycarbonyl)amino)octyl methanesulfonate (13.91 g, 0.043 mol, 3.0 eq) and tBuOK (5.15 g, 0.046 mol, 3.2 eq) leading to an almost complete orange/yellow solution. This mixture is then heated up to 80° C. turning into a thick solution. After 24 to the reaction mixture was reloaded 8-((tert-butoxycarbonyl)amino)octyl methanesulfonate (4.64 g, 0.014 mol, 1.0 eq) and tBuOK (1.93 g, 0.017 mol, 1.2 eq) and the solution was stirred at 80° C. The temperature was allowed to come back at 23° C. and the reaction mixture was hydrolyzed with a NH₄Cl saturated aqueous solution. Solution was diluted with water and EtOAc. Liquid/liquid extraction was performed and organic layer was washed once with water and brine, dried over Na2SO4, filtered, and concentrated under vacuum affording a brownish oil. Oily residue was taken up into a minimum of THE and purified by silica column chromatography using Heptane/EtOAc (90:10 to 80:20) as eluent. Purest fractions were combined and concentrated under vacuum affording the desired product (7.5 g, 0.007 mol, 48% yield) as a slight yellowish oil.

### Step 3 and 4:

In a 250 mL round bottom flask was introduced the previous product (6.60 g, 0.006 mol, 1.0 eq) followed by Dioxane (40.0 mL) leading to a yellowish solution. HCl 4M in Dioxane (7.6 mL, 0.030 mol, 5.0 eq) was added to this solution and it was stirred at 50° C. for 3 h. When complete conversion was observed by SFC, the reaction mixture was diluted with DIE and allowed to come back at 23° C., yielding to a suspension. The solid was triturated at 23° C. in the solvent mixture for 18 hours and then collected by filtration under N2, washed with DIE and dried at 45° C. under vacuum affording a yellowish solid (5.60 g, 0.006 mol). The yellow solid was transferred into an erlenmeyer and diluted into 2-MeTHF and a 1M NaOH aqueous solution. After complete solubilization, liquid/liquid extraction was performed, the organic layer was washed once with water and brine, dried over Na2SO4, filtered, and concentrated under vacuum affording the desired product (5.50 g, 0.006 mol, 87%) as a colorless oil containing 15% w residual 2-Me-THF.

### Step 5:

In a 250 mL three necked round bottom flask was introduced the previous product (5.50 g, 0.006 mol, 1.0 eq) and 2-cyano-3,3-diphenylacrylic acid (1.554 g, 0.006 mol, 1.0 eq) followed by THF (55.0 mL). To this solution Et3N (1.7 mL, 0.012 mol, 2.0 eq) was added. After 5 minutes T3P 50% in EtOAc (5.7 mL, 0.009 mol, 1.50 eq) was added and the reaction mixture was stirred at 23° C. for 1 h 15. The solution was diluted in 2-MeTHF and the organic layer washed with 1M NaHCO₃ aqueous solution, then with NH₄Cl aqueous saturated solution and finally with water and brine. The organic layer was then dried over Na2SO4, filtered and concentrated under vacuum affording 6.5 g as an off-white amorphous solid. The crude solid was taken up into DCM and purified by silica column chromatography (15-20 g silica/g product) using EtOAc/Heptane (50:50) as eluent and then DCM/MeOH (90:10). Purest fractions were concentrated under vacuum affording the desired product (2.50 g, 0.002 mol, 42% yield) as an off-white amorphous solid.

### Step 6 and 7:

In a 100 mL round bottom flask was introduced the previous product (2.40 g, 0.002 mol, 1.0 eq) and followed by 2-MeTHF (16.5 mL). At 23° C. methyl 2,5-dioxo-2,5-dihydro-1H-pyrrole-1-carboxylate (0.32 g, 0.002 mol, 0.95 eq) was added and the solution was stirred at 23° C. for 2 h. The reaction was hydrolyzed with NH₄Cl saturated aqueous solution and diluted with water and 2-MeTHF. Liquid/liquid extraction was performed and organic layer was washed once with water and brine, dried over Na2SO4, filtered and concentrated under vacuum affording 2.7 g as a yellowish oil. Oily residue was taken up into DCM and purified by silica column chromatography using first Heptane/EtOAc (40:60) and then DCM/MeOH (90:10) as eluent. Purest fractions were combined and concentrated under vacuum affording the intermediate product (1.30 g, 0.001 mol, 60% yield) as a colorless oil. In a 20 mL vial the previous intermediate product (1.03 g, 0.001 mol, 1.0 eq) was introduced followed by THF (8.0 mL) and H2O (4.0 mL). To this solution was added K2CO3 (0.11 g, 0.001 mol, 1.0 eq). The reaction was stirred at 23° C. for 3 h. The reaction was hydrolyzed with 0.2M HCl aqueous solution. Water and EtOAc were added and liquid/liquid extraction was performed. The organic layer was washed once with water and brine, dried over Na2SO4, filtered and concentrated under vacuum affording 1.1 g (18% EtOAc) as a yellowish oil. The crude product was purified by chromatography over a short silica pad eluting with Heptane/EtOAc (70:30 to 60:40). Purest fractions were combined and concentrated under vacuum, stripped with acetone affording the desired compound (0.950 g, 0.001 mol, 88% yield) as a yellowish oil.

¹H NMR (400 MHz, DMSO) δ 8.5 (d, J=8.9 Hz, 2H), 8.4 (t, J=5.7, 5.7 Hz, 1H), 8.1 (d, J=8.5 Hz, 2H), 7.6-7.4 (m, 3H), 7.4-7.3 (m, 5H), 7.1 (dd, J=7.4, 2.2 Hz, 2H), 7.1 (d, J=9.0 Hz, 2H), 7.0 (s, 2H), 6.8-6.5 (m, 4H), 4.1 (q, J=5.9, 5.8, 5.8 Hz, 4H), 4.0 (d, J=5.8 Hz, 4H), 3.9 (s, 3H), 3.3-3.3 (m, 2H), 2.9 (d, J=6.1 Hz, 2H), 1.9-1.7 (m, 6H), 1.6-1.2 (m, 22H), 1.2-1.0 (m, 1OH), 1.0-0.8 (m, 14H), 0.8-0.7 (m, 2H).

UV Absorbance Profil of Compound #6 (BOO-OM) Compound #6 (BOO-OM) and BEMT (6,6′-(6-(4-methoxyphenyl)-1,3,5-triazine-2,4-diyl)bis(3-((2-ethylhexyl)oxy)phenol)=Tinosorb S)+Octocrylene (OCR) were dissolved in a solvent at the same concentration (50 μM). Absorbance spectra was read with FLUOstar plate reader and results are shown in the following table 9.

TABLE 9

| | | |
|---|---|---|
| | BOO-OM λmax | BEMT + OCR λmax |
| Solvent | double peak | double peak |

| | | | | |
|---|---|---|---|---|
| Dottisol | 290 | 307 | 312 | 330 |
| Dubdis | 289 | 309 | 310 | 351 |

The λmax absorbance of the mix of BEMT-OCR shows a double peak at 310 and 351 nm when the λmax absorbance of the BOO-OM shifts by 20 nm and more in UVB showing a double peak at 289 and 309 nm at the same molar concentration diluted in Dubdis (diisopropyl sebacate). The same shift in UVB is obtained in Dottisol respectively 290 and 307 nm for BOO-OM and 312 and 330 nm for the mix of BEMT-OCR at the same molar concentration.

### Solubility of Compound #6 (BOO-OM) in Sunscreen Solvents:

### Material and Methods

Compound #6 (BOO-OM) and a sunscreen solvent were weighed in a Becher. The Becher was placed on a magnetic stir plate, heated up to 50° C., and the solution was vigorously mixed with a bar magnet.

BOO-OM is soluble in Dubdis (Diisopropyl sebacate, Stearinerie Dubois) and Dottisol (dimethyl isosorbide) at 50° C. at a ½ w/w and in Dimethyl capramide (Spectrasolv DMDA, Hallstar) at 50° C. at a 1/10 w/w.

### UV Protection Performance of Compound #6 (BOO-OM)

UVB protection has been assessed ex vivo on pig skin by measuring the autofluorescence quenching. BOO (Compound #5), BOO-OM (Compound #6) and the mix of BEMT+OCR were solubilized at the same molar concentration in DMDA (dimethyl capramide), covered 6.25 cm2 skin square and incubated 2 hours at 34° C., washed or not with 10 mL Ethanol for 5 min, and then 7 mm diameter skin punches were cut and placed in a black 96-well plate. UVB protection was measured after respectively an excitation at 300 nm and a fluorescence emission was read at 460 nm. The UV protection was measured after 2 hours of incubation at room temperature and results are shown in the following table 10.

TABLE 10
Autofluorescence of pig skin in UVB after 2 h of incubation
with BOO-OM (Mean +/− SD, N = 3)

UVB
Fluorescence (Ex300/Em460)

| | No Wash | EtOH Wash |
|---|---|---|
| OCR + BEMT | 49914 +/− 8563 | 152297 +/− 4564 |
| Compound #6 | 40608 +/− 3466 | 108875 +/− 4401 |
| (BOO-OM) | | |
| Compound #5 | 42968 +/− 3131 | 152417 +/− 17275 |
| BOO) | | |
| Blank | 199930 +/− 38803 | 204710 +/− 10214 |

The results show that UVB protection of the pig skin provided by compound #6 (BOO-OM) are similar compared to the mix OCR+BEMT.

After ethanol wash, UVB protection of the pig skin provided by the BOO-OM solution was reduced twice compared to the initial protection before washing. The same protocol applied on BOO, the non-bio adhesive control of BOO-OM, decreased three times the UVB protection measured prior to the washing. BOO-OM is bio adhesive whereas BOO is not.

### Bio Adhesion Performance on Thiolated Polylysine Matrix of Compound #5 (BOO) and Compound #6 (BOO-OM)

Polylysine and Polylysine enriched in SH by thiolation of primary amines using Traut's reagent were used for coating 96-well microplates. UV filters (BOO and BOO-OM) were incubated overnight under orbital agitation on the different matrices (n=3 for each condition) (uncoated wells were used as negative controls), in an ethanol/PBS (90%/10%) solution. After incubation, each microplate well was thoroughly washed 10 times with ethanol, before measuring UV absorbance. Whole UV/Visible absorbance spectrum from 220 to 1000 nm were measured in the center of each microplate well. In addition, specific UV absorbance at 310 nm was measured by matrix scan over the whole the well as a 10×10 matrix.

At 310 nm BOO-OM (BEMT-OCR-maleimide UV multifilter) showed a bio adhesion on thiolated polylysine matrix whereas BOO, the BEMT-OCR non bio adhesive multifilter, did not show a bio adhesion. Also, no bioadhesion of BOO-OM on polylysine matrix (non thiolated) was observed. These results show the bio adhesion of compound #6 (BOO-OM).

### Example 2. Preparation of a Composition Comprising a Compound of Formula (I)

100 g of a formulation comprising Compound #1 (SPF 15) are prepared as follows:
Part A: 8% of Compound #1 are dissolved in a mix of Cetiol B from BASF (di butyl adipate) 7% and Myritol 331 from BASF (cocoglycerides) 6% at 70° C. for 15 minutes.
Part B: 8% of SP Cithrol GMS 40 MBAL PA (SG) from Croda (Glyceryl monostearate) are mixed to 2% of SP Brij S2 MBAL SO (MV) from Croda (Steareth), 2% of Eumulgin B₁ from BASF (Ceteareth 12), 2% of Eumulgin B₂ from BASF (Ceteareth 20) at 60° C.
Part C: 1% of phenoxyethanol is added to 56% of purified water and warmed up at 60° C.
Part D: Part A is poured into part B at 60° C. and stirred to obtain a homogenous solution.
Part C is poured under stirring into Part D at 60° C., heating is stopped and the formulation is cooled down slowly under stirring.

The formulation is an oil-in-water solution. Emulsion droplets are homogenous and below 50 μm. The pH after 24 hours is between 5 and 7.

1-16. (canceled)

17. A compound represented by the following formula (I): 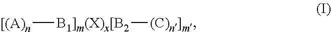
wherein:
X is an aryl, a heteroaryl, a polyol, a saccharide, an amino-acid, a peptide, or a polyamine;
each A represents independently a photoprotective moiety,
each B₁ and B₂ represents independently a linker,
C represents a functional group,
x is 0 or 1,
n is an integer from 1 to 12,
n′ is an integer from 1 to 2000,
m is an integer from 1 to 12,
m′ is an integer from 0 to 12, and
the number of photoprotective moieties A (m×n) is greater than or equal to 2.

18. The compound according to claim 17, wherein each photoprotective moiety A is independently bemotrizinol, diethylamino hydroxybenzoyl hexyl benzoate, bisdisulizole disodium, meradimate, terephtalylidene dicamphor sulfonic acid, oxybenzone, sulisobenzone, iscotrizinol, octinoxate, octisalate, octyltriazone, padimate O, homosalate, amiloxate, octocrylene, PEG-25 PABA, ensulizole, trolamine salicylate, cinoxate, benzophenone-9, dioxybenzone, avobenzone, enzacamene, bisoctrizole, diethylhexyl naphthalate, diethylhexyl syringylidene, tetramethylhydroxy piperidinol, sodium benzotriazolyl butylphenol sulfonate, benzotriazole dodecyl p-cresol sulfonate, butyloctyl salicylate, bis(cyano butylacetate) anthracenediylidene, dimethylcapramide, or ethyl hexyl methoxycrylene, ethyl hexyl triazone 2,4,6-tri([1,1′-biphenyl]-4-yl)-1,3,5-triazine, 2-(4,6-diphenyl-1,3,5-triazin-2-yl)-5-[(hexyl)oxy]-phenol, 2,4-Bis(2,4-dimethylphenyl)-6-(2,4-dihydroxyphenyl)-1,3,5-triazine, or 4-(4,6-Bis((biphenyl-4-yl)-1,3,5-triazine-2-yl)-1,3-benzodiole.

19. The compound according to claim 17, wherein:
x is 1, and
m′ is an integer from 1 to 12.

20. The compound according to claim 17, wherein each functional group C is independently selected from an aldehyde, an acetal, thiocetal, a thiol, a maleimide, a 5-methylene pyrrolone, a 3-bromo-5-methylene pyrrolone, a 5-hydroxy-pyrrolone, a 3,3-dimethylacrylic acid, a Mickael acceptor, a vinylsulfone, a disulfanylpyridine, a sulfonylaziridine, a vinylheteroarene, a vinylpyridine, a vinylpyrimidine, a vinyltriazine, a vinyltetrazine, an epoxide, a haloacetyl, an isocyanate, an isothiocyanate, a N-hydroxysuccinimide ester, a N-hydroxysulfosuccinimide ester, a hydroxy, an amino, an ammonium, a guanidinium, an imidocarbonate, a carboxylic acid, a carboxylic ester, an anhydride, a sulfonic acid, folic acid, biotin, streptavidin, avidin, antibodies, and single chain antibodies or fragments thereof, and derivatives thereof.

21. The compound according to claim 17, wherein:
x is 1, and
m′ is 0,
provided that at least two photoprotective moieties A are different.

22. The compound according to claim 17, wherein said radical X is a phenyl, a triazinyl, a glycerol or a pentaerythritol.

23. The compound according to claim 17, wherein:
x is 0, and
m′ is 0,
provided that at least two photoprotective moieties A are different.

24. The compound according to claim 17, wherein the linkers B₁ and B₂ are each independently a saturated or unsaturated, linear or branched carbon chain, an aliphatic or aromatic carbocycle, an aliphatic or aromatic heterocycle, a linear polymer, a branched polymer, a hyperbranched polymer, a dendrimer, or a residue thereof, and said linker optionally comprises at any one or both of its terminals a heteroatom independently chosen from —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO2—NH—, and —NH—SO₂—.

25. The compound according to claim 24, wherein the linkers B₁ and B₂ are each independently interrupted by:
one or more heteroatomic groups selected each independently from —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO2—NH—, and —NH—SO₂—, and/or
one or more heterocycloalkyls.

26. The compound according to claim 17, wherein the linkers B₁ and B₂ are each independently of formula (II): 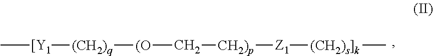
wherein:
Y₁ is selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO2—NH—, and —NH—SO₂—;
q is an integer from 0 to 35;
with the proviso that, when Y is —O—, q is different from 0;
p is an integer from 0 to 250; and
p+q is different from 0; or
Y₁—(CH₂)_{q} may form a heterocycle selected from the group consisting of a pyrrolidinyl and piperidinyl; and
p is 0;
Z₁ is selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO2—NH—, and —NH—SO₂—;
s is an integer from 0 to 6; and
k is an integer from 1 to 4.

27. The compound according to claim 17, wherein the linkers B₁ and B₂ are each independently of formula (III): 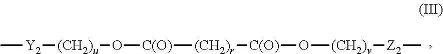
in which,
Y₂ and Z₂ are each independently selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO2—NH—, and —NH—SO₂—;
u and v are independently an integer from 1 to 50, and
r is an integer from 1 to 28.

28. The compound according to claim 17, wherein the linkers B₁ and B₂ are each independently of formula (IV): 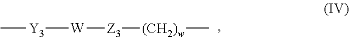
Y₃ and Z₃ are each independently selected from a single bond, —O—, —NH—, —S—, —SO₂—, —C(O)—, —O—C(O)—, —C(O)—O—, —C(O)—NH—, —NH—C(O)—, —NH—C(O)—O—, —O—C(O)—NH—, —SO2—NH—, and —NH—SO₂—;
W is an aryl or a heteroaryl; and
w is an integer from 1 to 150.

29. The compound according to claim 17, wherein said compound is selected from the group consisting of:
2-[(2-cyano-3,3-diphenyl-prop-2-enoyl)amino]ethyl 2-[4-(diethylamino)-2-hydroxy-benzoyl]benzoate;
8-[[2-[4-(diethylamino)-2-hydroxy-benzoyl]benzoyl]amino]octyl 2-cyano-3,3-diphenyl-prop-2-enoate;
8-[4-(4,6-diphenyl-1,3,5-triazin-2-yl)-3-hydroxy-phenoxy]octyl 2-hydroxybenzoate;
7-[4-[4,6-bis[4-(2-ethylhexoxy)-2-hydroxy-phenyl]-1,3,5-triazin-2-yl]phenoxy]heptyl 2-(4-amino-2-hydroxy-benzoyl)benzoate;
8-[4-(4,6-diphenyl-1,3,5-triazin-2-yl)-3-hydroxy-phenoxy]octyl 2-cyano-3,3-diphenyl-prop-2-enoate;
2-[2-[2-[4-[4,6-bis[4-(2-ethylhexoxy)-2-hydroxy-phenyl]-1,3,5-triazin-2-yl]phenoxy]ethoxy]ethoxy]ethyl 2-cyano-3,3-diphenyl-prop-2-enoate;
N-[7-[4-[4,6-bis[4-(2-ethylhexoxy)-2-hydroxy-phenyl]-1,3,5-triazin-2-yl]phenoxy]heptyl]-2-hydroxy-benzamide;
6-[[2-[4-(diethylamino)-2-hydroxy-benzoyl]benzoyl]amino]hexyl 2-hydroxybenzoate;
5-[5-(2-ethylhexoxy)-2-[4-[4-(2-ethylhexoxy)-2-hydroxy-phenyl]-6-(4-methoxyphenyl)-1,3,5-triazin-2-yl]phenoxy]pentyl 2-cyano-3,3-diphenyl-prop-2-enoate;
N-[5-[4-[4,6-bis(2,4-dimethylphenyl)-1,3,5-triazin-2-yl]-3-hydroxy-phenoxy]pentyl]-2-[4-(diethylamino)-2-hydroxy-benzoyl]benzamide;
[(3Z)-3-[[4-[(Z)-[4-[7-[2-(2-hydroxy-4-methyl-benzoyl)benzoyl]oxyheptylsulfamoylmethyl]-7,7-dimethyl-3-oxo-norbornan-2-ylidene]methyl]phenyl]methylene]-7,7-dimethyl-2-oxo-norbornan-1-yl]methanesulfonic acid;
N-[5-[4-[4,6-bis(4-phenylphenyl)-1,3,5-triazin-2-yl]-3-hydroxy-phenoxy]pentyl]-2-[4-(diethylamino)-2-hydroxy-benzoyl]benzamide;
5-[(2-phenyl-3H-benzimidazol-5-yl)sulfonylamino]pentyl-2-[4-(diethylamino)-2-hydroxy-benzoyl]benzoate;
4-[9-[6-(2-cyano-3,3-diphenyl-prop-2-enoyl)oxyhexyl]-2,4,8,10-tetraoxaspiro[5.5]undecan-3-yl]butyl 2-[4-(diethylamino)-2-hydroxy-benzoyl]benzoate;
[(3Z)-3-[[4-[(Z)-[4-[3-[6-[2-(2-hydroxybenzoyl)oxyethoxy]-6-oxo-hexanoyl]oxypropylsulfamoylmethyl]-7,7-dimethyl-3-oxo-norbornan-2-ylidene]methyl]phenyl]methylene]-7,7-dimethyl-2-oxo-norbornan-1-yl]methanesulfonic acid;
O6-[3-(2-cyano-3,3-diphenyl-prop-2-enoyl)oxypropyl]O1-[3-[2-[4-(diethylamino)-2-hydroxy-benzoyl]benzoyl]oxypropyl]hexanedioate;
[(3Z)-3-[[4-[(Z)-[4-[3-[6-[2-[(5-benzoyl-4-hydroxy-2-methoxy-phenyl)sulfonylamino]ethoxy]-6-oxo-hexanoyl]oxypropylsulfamoylmethyl]-7,7-dimethyl-3-oxo-norbornan-2-ylidene]methyl]phenyl]methylene]-7,7-dimethyl-2-oxo-norbornan-1-yl]methanesulfonic acid;
2-[[4,6-bis[4-(2-cyano-3,3-diphenyl-prop-2-enoyl)oxybutylamino]-1,3,5-triazin-2-yl]amino]ethyl-2-(2-hydroxy-4-methyl-benzoyl)benzoate;
2-[[4-[2-(2,5-dioxopyrrol-1-yl)ethoxy]-6-[2-[(2-hydroxybenzoyl)amino]ethylamino]-1,3,5-triazin-2-yl]amino]ethyl 2-[4-(diethylamino)-2-hydroxy-benzoyl]benzoate;
N-[2-[[4-[5-(2,5-dioxopyrrol-1-yl)pentoxy]-6-[2-[2-[2-[2-[[rac-(3Z,4R)-7,7-dimethyl-2-oxo-3-[[4-[rac-(Z)-[rac-(1R)-4-ethyl-7,7-dimethyl-3-oxo-norbornan-2-ylidene]methyl]phenyl]methylene]norbornan-1-yl]methylsulfonylamino]ethoxy]ethoxy]ethoxy]ethylamino]-1,3,5-triazin-2-yl]amino]ethyl]-2-hydroxy-benzamide;
2-hydroxy-N-[2-[[4-[5-(2-methylene-5-oxo-pyrrol-1-yl)pentoxy]-6-[2-[2-[2-[2-[[rac-(3Z,4R)-7,7-dimethyl-2-oxo-3-[[4-[rac-(Z)-[rac-(1R)-4-ethyl-7,7-dimethyl-3-oxo-norbornan-2-ylidene]methyl]phenyl]methylene]norbornan-1-yl]methylsulfonylamino]ethoxy]ethoxy]ethoxy]ethylamino]-1,3,5-triazin-2-yl]amino]ethyl]benzamide;
2-[[4-[4-[2-(2,5-dioxopyrrol-1-yl)ethoxy]anilino]-6-[2-[(2-hydroxybenzoyl)amino]ethyl amino]-1,3,5-triazin-2-yl]amino]ethyl 2-[4-(diethylamino)-2-hydroxy-benzoyl]benzoate;
2-[[4-[4-[2-(2,5-dioxopyrrol-1-yl)ethoxy]phenyl]-6-[2-[(2-hydroxybenzoyl)amino]ethyl amino]-1,3,5-triazin-2-yl]amino]ethyl 2-[4-(diethylamino)-2-hydroxy-benzoyl]benzoate;
6-[[2-[4-(diethylamino)-2-hydroxy-benzoyl]benzoyl]amino]hexyl 2-cyano-3,3-diphenyl-prop-2-enoate;
2-cyano-N-(8-(5-((2-ethylhexyl)oxy)-2-(4-(4-((2-ethylhexyl)oxy)-2-hydroxyphenyl)-6-(4-methoxyphenyl)-1,3,5-triazin-2-yl)phenoxy)octyl)-3,3-diphenylacrylamide; and
2-cyano-N-(8-(2-(4-(2-((8-(2,5-dioxo-2,5-dihydro-1H-pyrrol-1-yl)octyl)oxy)-4-((2-ethylhexyl)oxy)phenyl)-6-(4-methoxyphenyl)-1,3,5-triazin-2-yl)-5-((2-ethylhexyl)oxy)phenoxy)octyl)-3,3-diphenylacrylamide.

30. A composition comprising at least one compound of formula (I) as defined in claim 17 and at least one excipient.

31. The composition according to claim 30, wherein said composition is a sunscreen composition, a cosmetic composition, or a topical composition in the form of a suspension, a cream, a spray, an aerosol, a butter, a stick, a gel, an ointment, a lotion, a solution, a solid, an emulsion, a micro-emulsion, an oil, a lyophilizate, a milk, a powder, a paste, a wax, a mousse, a patch, a film, a micelle, a liposome, or a foam.

32. A kit comprising:
a composition as defined in claim 30,
a washing composition, and
optionally an instruction guide.

33. A material comprising a support and at least one compound as defined in claim 17, said support being a natural or synthetic polymeric support, a natural or synthetic fiber support, a stone, a metal, a plastic, a rubber, a glass support, or a paint.
